(19) United States
(12) Patent Application Publication
(10) Pub. No.: US 2021/0315512 A1
(43) Pub. Date: Oct. 14, 2021

(54) METHOD AND SYSTEM FOR COSMETIC RECOMMENDATIONS

(71) Applicant: SKIN REJUVENATION TECHNOLOGIES (PTY) LTD, Irene, Centurion (ZA)

(72) Inventors: Markus DEPFENHART, Hamburg (DE); Leandri VAN DER WAT, Johannesburg (ZA)

(21) Appl. No.: 17/260,967

(22) PCT Filed: Jul. 16, 2019
(86) PCT No.: PCT/IB2019/056063

§ 371 Date: Jan. 15, 2021

(30) Foreign Application Priority Data
Jul. 16, 2018 (ZA) 201804725

(51) Int. Cl. A61B 5/00; G16H 50/30; G16H 20/10; G06T 7/00; G16B 20/20

(52) U.S. Cl. CPC A61B 5/442; G16H 50/30; G16H 20/10; G06T 2207/30088; G16B 20/20; G06T 2207/20021; G06T 7/0012

(57) ABSTRACT

There is provided a cosmetic method for scoring and assigning codes for a person's skin. The cosmetic method involves dividing a person's skin into a plurality of zones; evaluating zones predetermined skin characteristics in each zone; scoring the zones on a predetermined scale of scores for each predetermined skin characteristic based on the evaluation; determining a collective score wherein the scores for each zone for the predetermined skin characteristic is combined; and assigning a skin status quo code by utilising the collective score for each predetermined skin characteristic.

### TECHNICAL FIELD

This disclosure relates generally to a field of skin care, in particular, the disclosure relates to a cosmetic method and system of scoring a person's skin, a cosmetic product and genetic profile and utilising the scoring to customize a cosmetic intervention.

### BACKGROUND ART

Many methods are currently used to improve skin quality, health or appearance. To a large extent, the use of skin care products is based on individual preference or when recommended by a professional are generic in nature. Generally, it has become increasingly desirable to customize cosmetics for an individual. Skin health and appearance focus has shifted in the last couple of years to focus on customizing topical treatments in cosmetology. Skin ageing is a growing concern and aesthetic interventions have been attempted for individual skin concerns.

The applicant is aware of various attempts to profile an individual's skin based on biophysical parameters and using genetic profiling techniques.

Dermalogica has implemented a face mapping based on 11 different zones of the face, wherein each zone is analysed for a skin characteristic https://www.dermalogica.com/face-mapping-skin-analysis/ys_featured_5, default,pg.html. This method discloses a qualitative mapping by a practitioner utilizing a zone based mapping to come to a subjective recommendation and the assessments cannot be tracked or validated and does not create a skin or face profile,

IPSA has implemented a skin analysis system and the IPSALYSER™ skin diagnosis system discloses analysis of 10 categories of Skin Balance, Original Skin Tone Contrast, Translucency, Transparency, Facial Texture Comparison, Moisture Retention Capability, Sebum Secretion, Firmness and Elasticity Test, Keratin Metabolism and Rosiness in order to recommend one of their products based on the skin analysis conducted. The method discloses a system and a self-questionnaire based system for one- or a two-skin characteristic based on patient concerns and is not holistic, quantitative nor reproducible.

HelloAva has implemented a 12 question and multiple-choice answer system within a “skin matching app” to make product recommendations of multiple products across multiple brands for a skin care regime (https://www.allure.com/story/hello-ava-app). The system relies on self-directed questionnaire basis by individuals and does not have a systematic skin analysis system to create a skin profile for an individual.

Clinique has implemented a computer aided questionnaire based system for a customer (https://www.clinique.co.uk/Diagnostics) which allows a customer to select up to two skin concerns and directs the individual customer to the company's product(s) that matches the customer's skin concern. This system does not take into account a large range of individual skin characteristics based on skin aging or photo-damage and is subjective, relies on a few self-identified concerns, lifestyle based only and is not comprehensive in terms of a skin profile for the individual.

Jain et. al., (2017) Journal of Cosmetic Dermatology, 16: 132 -143; reveal a global ranking scale (GRS) established by Galderma, for assessing skin and looks at nine aspects of facial appearance with four domains cited as skin quality, wrinkles, morphology and volume and are graded on a four-point scale of 0 denoting none to 4 denoting severe. The method in this disclosure of assessment of skin concerns is subjective, lacks a comprehensive and empirical approach and, is not a validated assessment of potential treatment outcomes.

Lakhani et. al. (2016) IOSR Journal of Dental and Medical Sciences, Vol 15, Issue 7: 89-99; discloses gradings available in prior art for individual skin conditions utilising clinical skin parameters for medical diagnosis and is not directed to creating a skin profile.

The disclosure in Luc Levy et. al., (2004); Medical Laser Application 19(4): 223-229, is directed to various state of the art evaluation techniques for individual skin characteristics as is currently available in the field of cosmetic dermatology and the disclosure does not reveal a skin profile creation, based on the evaluation techniques disclosed.

Skin ageing is a growing concern and aesthetic interventions have been attempted for individual skin concerns by measuring various genetic parameters to assess intrinsic ageing. Trojahn et. al., (2015) BioMed Research International, Vol 2015, Article ID 318586, 9 pages; disclose that extrinsic ageing causes various changes in skin elasticity, wrinkling, sagging and yellowness in a customer. The methods in this disclosure assesses individual characteristics qualitatively and do not produce an overall skin profile for an individual that is systematically and efficiently utilizable to estimate skin characteristics and a skin profile for an individual.

US Patent application No. 2011/0305737 A1, filed on 7 Jun. 2011; entitled “Multi-active microtargeted anti-aging skin cream polymer technology, discloses a cosmetic formulation with a high number of active ingredients directed to but not limited to wrinkles, pigmentation namely brown spots due to aging of the skin, also able to decrease redness, acne and rosacea blemishes. The disclosure further reveals a grading scale employed for the characteristics of skin irritation on a scale of 0 to 4 and a scale of 0 to 7 for the grading of the characteristic of crow's feet lines/wrinkles.

PCT Patent application No. PCT/GB2012/052725 (Publication No. WO2013/093407A1) filed on 1 Nov. 2012 entitled “Product selection using genetic analysis” discloses a method of assessing a sample of genetic material from an individual for the presence or absence of single nucleotide polymorphisms (SNPs), associating the SNPs for effectiveness to an active in a cosmetic product. They disclose associating a weight for the SNP locations with an active and recommendation of a cosmetic product with those actives to affect the SNPs.

US Patent application US2016/0068904A1 discloses a method of creating an individual's skin profile based on specific single nucleotide polymorphisms (SNPs) as a genetic marker and scoring actives to recommend a personalized skin care regime based on the genetic score, as determined by the method in the disclosure, of the individual.

U.S. Pat. No. 6,782,307 discloses the system of utilising an individual's skin image for an internet based or kiosk system that analyses skin appearance on a qualitative level and a customized topical formulation is created for the individual customer in a manufacturing facility on demand.

U.S. Pat. No. 5,622,692 discloses a method and system directed to customizing facial foundation products. The system profiles the skin characteristics at a qualitative level and aids in adjusting the colour of premixed foundations for the individual customer and does not address any skin characteristics.

PCT Application No. PCT/IB2012/057558 (Publication No. WO2013098531 A1) entitled “Method for delivering cosmetic advice” discloses a method to identify indications of the skin related to skin colour and cosmetic advice related to it. The invention requires that a colour image is captured from a region, or a body of an individual and the image has a colour reference chart in the field of the image, which is then utilised to determine a skin type, a skin colour relating to a pigmentation or a depigmentation process and appropriate photoreaction, or a product that may be applied to temporarily modify the colour of the individual's skin. This disclosure is narrowly directed to a determination of the skin colour and possible pigmentation or depigmentation, and products related thereto.

Wrinkle severity has been quantified by a number of systems including Hamilton's Classification, Glogau's classification and Fitzpatrick's classification. It is understood that many wrinkle severity scales have been developed to reproducibly classify wrinkle severity and improvement after professional treatment. However, these scales are merely on the basis of wrinkle appearance on the skin only and do not consider the holistic condition of the skin.

It is an object of the present disclosure to provide for an empirical and systematic analysis of skin characteristics and a method and system thereof, which will, in the Inventors' view, address some of the above-mentioned drawbacks. None of the above disclosures, taken singly or in combination, describes the present invention as revealed.

### SUMMARY OF INVENTION

According to an aspect, there is provided a cosmetic method, the method comprising:
dividing at least one area of a person's skin into a plurality of zones;
evaluating at least one of the plurality of zones for at least a predetermined skin characteristic in each zone;
scoring the zones on a predetermined scale of scores for each predetermined skin characteristic based on the evaluation;
determining a collective score wherein the scores for each zone for the predetermined skin characteristic is combined; and
assigning a skin status quo code by utilising the collective score for each predetermined skin characteristic.

A preferable method of assigning a status quo code of the cosmetic method above is scoring the zones on a predetermined scale of scores for each of the predetermined skin characteristics to determine a zone score, which is a collective code of each of the scores of each of the predetermined areas within a zone; and
assigning a skin status quo code by cumulating all zone scores as an overall severity ranking for the predetermined skin characteristic.

The person's skin may be a face, neck and a chest.

The zones may vary in number from 1 to 15. The preferable number of zones may be 5.

At least one or more of the predetermined skin characteristics may be selected from the group consisting essentially of a characteristic of wrinkles, a characteristic of dryness, a characteristic of sensitivity and a characteristic of pigmentation.

The step of evaluating the predetermined skin characteristics may be based on a plurality of skin analysis techniques.

According to another embodiment a cosmetic method, the method further comprising:
choosing a plurality of actives in a cosmetic product that has at least one remedial mode of action for at least a predetermined skin characteristic;
determining a score from a range of predetermined scale of scores, for at least one of the plurality of actives in the cosmetic based on the remedial mode of action of the active;
combining the scores of each of the actives based on the remedial mode of action for the predetermined skin characteristic to assign a combined score for the predetermined skin characteristic; and
assigning a cosmetic code for the cosmetic based on the combined scores and directed to the at least one predetermined skin characteristic.

According to the method the plurality of predetermined actives having remedial modes of action for the characteristics of wrinkles, dryness, sensitivity and pigmentation may be selected from the group essentially consisting of anti-acne agents, antimicrobial agents, anti-inflammatory agents, analgesics, antietythemal agents, antipruritic agents, antiedemal agents, antipsoriatic agents, antifungal agents, skin protectants, Sunscreen agents, Vitamins, antioxidants, Scavengers, anti-irritants, antibacterial agents, antiviral agents, anti-aging agents, protoprotection agents, hair growth enhancers, hair growth inhibitors, hair removal agents, antidandruff agents, anti-Seborrheic agents, exfoliating agents, wound healing agents, anti-ectoparacitic agents, anti-pigmentation agents, whiteners, moisturizing agents, Sebum modulators, immunomodulators, hormones, botanicals, moisturizers, astringents, cleansers, Sensates, antibiotics, anesthetics, Steroids, tissue healing Substances, tissue regenerating Substances, amino acids, peptides, minerals, ceramides and biohyaluronic acids.

According to another embodiment, a cosmetic method, the method comprising:
utilising the skin status quo code of the method above;
determining the at least one predetermined skin characteristic that may benefit from a cosmetic intervention; and
recommending at least one cosmetic intervention to effect the predetermined skin characteristic.

The cosmetic intervention of the above method may be a cosmetic code based product.

According to another embodiment of the cosmetic method, the method further comprising:
determining an intrinsic code of the person's skin for the predetermined skin characteristics, wherein the intrinsic code is derived by a method of assigning scores for polymorphisms based on a priority code assigned to a presence or absence of a plurality of predetermined polymorphisms which are known to effect the predetermined skin parameters selected from the group essentially consisting of MMP1, COL1A1, MC1R, ASIP, XRCC1, hOGG1, TERT, SOD2, GPX1, NQO1, GSTP1, EPHX1, IL6, IL6-R, CRP and TNFA genes;
comparing the intrinsic code, which is a measure of the effect of a person's genetics on the person's skin for predetermined skin characteristics, to the skin status quo to create a second unique code, which reveals an effect on predetermined skin characteristics that are not effected by the genetics of the person; and
recommending a cosmetic intervention which may be a cosmetic directed to the second unique code to effect the overall skin status quo code, wherein the cosmetic code of the cosmetic is directed to effect at least one of the predetermined skin characteristics.

According to an embodiment, there is provided a cosmetic system, the system comprising:
at least one processor; and
a computer readable storage medium having stored databases and instructions therein which, when executed by the processor, cause the processor to perform operations comprising any of the method steps defined above.

In an embodiment of the system, the processor may comprise a skin-scoring module and a cosmetic analyser.

A further embodiment may include a genetic scoring module.

The skin-scoring module of the system may comprise a first input interface adapted to receive data of a plurality of predetermined skin characteristics.

The first input interface may be adapted to receive a direct data input, data derived from analysis of a photographic image or video images of a zone or an area of a person's body in respect of at least one of a plurality of skin characteristics.

The first input interface may be further adapted to process a photographic image or video images to derive data and receive the data as input.

The skin-scoring module may further comprise a means for evaluating the data received by the first interface in relation to a weighting table for at least one of a plurality of predetermined skin characteristics;
scoring the areas of the body or preferably the zones on a predetermined scale of scores for each skin characteristic;
determining a collective score, wherein the scores for each zone or area of the body for the predetermined skin characteristic is combined; and
assigning a status quo code by utilising the collective score for each predetermined skin characteristic.

The skin-scoring module may execute the processing and assessment of the data received by the first input interface on the basis of a weighting of scores in a weighting table database for the predetermined skin characteristic.

The cosmetic analyser may comprise a second input interface adapted to receive data related to a plurality of actives in a cosmetic product.

The cosmetic analyser may further comprise a means for evaluating data received by the second input interface for the plurality of actives in a specific cosmetic product for at least one remedial mode of action of the active for at least a predetermined skin characteristic;
determining a score, from a range of predetermined scale of scores, for at least one of the plurality of actives in the cosmetic based on the remedial mode of action of the active;
combining the scores of each of the actives based on the remedial mode of action for the predetermined skin characteristic to assign a combined score for the predetermined skin characteristic; and
assigning a cosmetic code for the cosmetic based on the combined scores and directed to the at least one predetermined skin characteristic; and
deriving a cosmetic code for a cosmetic product; based on the data received by the second input interface and a weighted actives database.

The means for evaluating the data received by the second input interface for the plurality of actives in a specific cosmetic product may utilise data from a weighted actives database, which is stored in the computer readable storage medium.

In another embodiment the processor may further comprise a genetic scoring module for the method of deriving an intrinsic code.

The genetic scoring module may comprise a third input interface adapted to receive data related to a genetic polymorphism profiling.

The genetic scoring module may further comprise a means for deriving an intrinsic code for a person based on the data input in the third input interface and a polymorphism-weighted database stored in the system.

The third input interface may be adapted to receive data from an analysis of a person's genetic profile.

According to another embodiment of the cosmetic system, the genetic scoring module comprising:
a means for determining an intrinsic code of the person's skin for the predetermined skin characteristics, wherein the intrinsic code is derived by assigning specific scores for a plurality of predetermined skin characteristics from data obtained in the third input interface and may utilise a polymorphisms weighting table to understand risks associated to a specific polymorphism;
a means for comparing the intrinsic code to the skin status quo code, utilising a predictive equation directed to deriving a second unique code, which is a measure of scores of predetermined skin characteristics other than inherent genetics of a person and its influence on the skin's appearance and health.

According to another embodiment, the system may comprise a further processor, which is recommendation module.

The recommendation module may be adapted to receive a plurality of data related to skin status quo code, an intrinsic code and cosmetic code.

The recommendation modules may comprise means for integrating data derived from the skin scoring module, genetic scoring module and cosmetic analyser.

The recommendation module may include a means for analyzing the skin status quo code derived from the execution of the system above, executing a predictive equation, for determining the at least one predetermined skin characteristic that may benefit from a cosmetic code based cosmetic product; and a further means for integrating a recommendation of a cosmetic product with a specific cosmetic code to effect at least one predetermined skin characteristic.

The computer readable storage may comprise databases which may include a plurality of weighting tables; and a set of instructions directed to at least one of a plurality of predictive equations for scoring a skin status quo code, a intrinsic code, a cosmetic code, a second unique code or a set of instructions to utilise a selection of the codes to make a cosmetic recommendation.

The databases may include the a weighting of scores in a weighting table database for a set of predetermined skin characteristics, the weighted actives database, the polymorphism-weighted database, a further weighting table, which maybe a database of the cosmetic products wherein the cosmetic coded products are weighted on the basis of its capacity to effect a predetermined skin characteristic.

The recommendation module may use a means for integrating data and may be a software application directed to comparing and analyzing the skin status quo code to the weighting table of the database of the cosmetic products as per the cosmetic code, to match a cosmetic code based cosmetic product.

The means for integrating data may be a software application directed to deriving a second unique code, by comparing and assessing the differences between the skin status quo code and the intrinsic code, and further matching a cosmetic code based cosmetic product.

The recommendation module may comprise a means for creating a profile.

The profile may be the codes derived for a person and may include the skin status quo, an intrinsic code and a second unique code.

The profile may further comprise results of the comparative analysis of the skin status quo code to the second unique code to determine a potential recommendation for a person.

The computer readable storage medium may further comprise a database of profiles and a set of instructions directed to utilising a profile derived at a first time point; and comparing the profile derived at a first time point to a second profile derived at a second time point.

The recommendation module may comprise a means to utilise the profile to recommend a treatment plan for the person.

The treatment plan may comprise a cosmetic product recommendation.

The recommendation module may further comprise a means to output the recommendation to a user.

The computer directed storage medium may be further adapted to store and integrate the recommendation to a profile.

### BRIEF DESCRIPTION OF DRAWINGS

The invention will now be further described in more detail, by way of example only, with reference to the accompanying diagrammatic drawings.

In the drawings:

FIG. 1 is a flow diagram illustrating in general terms a method of assigning a skin status quo code for an individual;

FIG. 2 is a diagrammatic representation of a person's face divided into multiple zones;

FIG. 3 is a diagrammatic representation of a person's face showing the various areas of the face for analysis of the skin characteristic of wrinkles;

FIG. 4A-4B is an example method of a scoring scale for the skin characteristic of wrinkles;

FIG. 5A is an example method of a scoring scale for the skin characteristic of sensitivity;

FIG. 5B is a visual skin guide for scoring the skin for the characteristic of sensitivity;

FIG. 6 is an example method of a scoring scale for the skin characteristic of pigmentation;

FIG. 7 is an example method of a scoring scale for the skin characteristic of dryness;

FIG. 8 shows an example data structure-detailing data/information utilised in the analysis of intrinsic skin code;

FIG. 9 is a flow diagram illustrating in general terms a method of assigning a cosmetic code for a cosmetic product;

FIG. 10A is an illustrative guide for the scoring of actives within a cosmetic product;

FIGS. 10B, 100, 10D and 10E are illustrative examples of scoring of the actives within cosmetic products to derive cosmetic codes;

FIG. 11 a flow diagram illustrating in general terms a method of matching a cosmetic intervention for a person using only the skin status quo code and the cosmetic code;

FIG. 12 is a flow diagram illustrating in general terms a method of assigning an intrinsic score;

FIGS. 12A Is an illustrative guide to scoring as per the method of FIG. 12;

FIG. 12C is an illustrative example of scoring for a particular human subject A to derive the subject's intrinsic code;

FIG. 13 is a diagrammatic illustration in general terms for a cosmetic system;

FIG. 14 illustrates a system in general terms for recommending a cosmetic product using the skin status quo code and the cosmetic code; and

FIG. 15 illustrates a system in general terms for matching a cosmetic product using the skin status quo code, intrinsic code and the cosmetic code.

Wherever possible, the same reference numbers will be used throughout the drawings to represent the same steps or modules.

### DESCRIPTION OF EMBODIMENTS

While various inventive aspects, concepts, and features of the disclosure may be described and illustrated herein as embodied in combination in the exemplary embodiments, these various aspects, concepts and features may be used in many alternatives embodiments, either individually or in various combinations and sub-combinations thereof. Unless expressly excluded herein all such combinations and sub-combinations are intended to be within the scope of the present invention. Still further, while various alternative embodiments as to the various aspects, concepts, and features of the invention—such alternative structures, configurations, methods, circuits, devices and components, software, hardware, control logic, alternatives as to form, fit and function, and so on may be described herein, such descriptions are not intended to be a complete or exhaustive list of available, alternative embodiments, whether presently known or later developed.

Although specific advantages have been enumerated in this disclosure, various embodiments may include some, none, or all of the enumerated advantages. Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below. Unless otherwise specifically noted, articles depicted in the drawings are not necessarily drawn to scale.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

“Skin characteristics” typically refers to a feature or quality belonging to a person and as such varies across the population. This would be something to the effect of a facial feature and is not medically associated to a disease/infection/ailment and can be improved upon but not resolved. The term is used interchangeably include terms “skin parameters”, “skin conditions”, “parameters” and “conditions” in the context of this specification. A skin condition although typically refers to an illness or a medical problem, includes ailments and inflammatory disorders for example psoriasis, eczema and rosacea, its use in this specification does not include ailments and inflammatory disorders.

Cosmetic interventions are generally known in the art and may be invasive or non-invasive aesthetic treatments and also include cosmetic products.

Wrinkles is a skin characteristic defined to include wrinkles, sagging and the parameter of volume loss in the context of this disclosure and in this specification has also been denoted as “W”.

Dryness is a skin characteristic and defined as the presentation of flakiness, lack of radiance, tightness (loss of pliability) and rough texture and is denoted as “D”.

Sensitivity is a skin characteristic and defined to include perceivable inflammation and redness in the skin and in this specification may also be denoted as “S”.

Pigmentation is a skin characteristic and describes the amount of unwanted pigmentation presenting in the skin. This characteristic is assessed by general evenness of the skin's appearance, including considerations of both hyper- and hypo-pigmentation and is also denoted as “P”.

Polymorphism refers to a change or variation in the sequence of a gene, DNA or RNA sequence or chromosome. The change or variation can be as a result of a substitution (transition or transversion mutation) or an insertion/deletion, although not limited to these terms. These variations may or may not result in synonymous or non-synonymous amino acid changes and may be present in noncoding genomic regions.

Single nucleotide polymorphism (SNP) refers to a variation at a single position in a DNA or RNA sequence. In the presence of a polymorphism, the wild type state (reference gene or original state) of the gene is altered to a heterozygous or homozygous alternative. Polymorphism can refer to single variations or multiple variations that are either closely spaced, consecutive or spread out and non-consecutive along the residues.

Skin category refers to phenotypic features that are affected by the presence or absence of polymorphisms. Skin categories include, and are not limited to i) firmness and elasticity, ii) sun sensitivity and pigmentation, iii) sun damage, protection and repair, iv) protection from oxidative stress, v) detoxification and vi) inflammation.

This disclosure reveals a method and system of comprehensive evaluation of an individuals skin characteristics to determine a skin profile by assigning a skin status quo code and an intrinsic code for an individual. A further embodiment reveals a method assigning a cosmetic code based on actives for a cosmetic product, and recommending cosmetic interventions based on the skin status quo code and intrinsic code for the individual.

An embodiment of the disclosure includes cosmetic intervention recommendations, which may also include an appropriate cosmetic product, based on its cosmetic code directed to improving the skin status quo code and may take into account the intrinsic code.

An embodiment of this disclosure is directed to evaluating the skin systematically to recommend cosmetic interventions, which are designed to improve the appearance of defined skin characteristics.

A further desirable feature of this disclosure is that the skin status quo code is compared to a cosmetic code which has been determined for a cosmetic product, according to its active ingredient contents, in order to make a skin care recommendation.

Prior art teaches skincare cosmetic interventions for symptomatic treatments. An object of this present disclosure is a systematic and empirical approach to evaluation of one or a multiple set of skin characteristics, which are reproducible and not subjective to determine a skin profile for an individual.

A skin profile is arrived at by evaluating chosen skin characteristics and reveals skin health, which is beyond a mere symptomatic appearance of the skin. Also a person's skin appearance and health is not a static expression of genetic predispositions, and what is seen on the skin is a combination of genetic and external factors. These external factors are related to various factors including diet, hormone levels, personal hygiene and habits, environmental factors including UV exposure from the sun. A reference to “profile” or “skin profile” in this disclosure relates to either one or a combination of the skin status quo code, intrinsic code, a second unique code derived from comparing the skin status quo code and the intrinsic code to indicate skin health.

Advances in DNA analysis specifically targeted towards the skin are well known and been documented extensively in prior art. The method and system of the present disclosure is able to utilise a variety of polymorphisms or single nucleotide polymorphisms (SNPs) known to affect the skin as a tool to derive an understanding of inherent factors that affect the skin's health to create a genetic score code in a unique pattern representing predetermined skin characteristics, termed as the intrinsic code and this unique representation pattern is new. SNPs that affect the skin categories for loss of firmness, sun sensitivity and pigmentation, sun damage, protection and repair, detoxification systems and sensitivity, protection from oxidative stress and inflammation are profiled and utilised to derive the intrinsic code which represents the skin's tendency towards these specified alterations.

The scoring method and system of this disclosure is new as a language that communicates the individual's skin status based on a set of predetermined skin characteristics that are evaluated and scored and its use in cosmetic interventions on how the skin's health and appearance can be improved.

A zone based systematic evaluation and scoring of each predetermined skin characteristic makes the method in this disclosure a reproducible and objective technique for skin profiling and is new. This systematic evaluation and the scoring provides for an empirical and validated method, which can be used across a period to assess a pre- and post-treatment effect.

In a preferred embodiment of this disclosure four skin characteristics of wrinkles, sensitivity, pigmentation and dryness are evaluated in each zone, and a scale of scores, which are ranked for severity of presence of the skin characteristic from 1-5, are assigned for each zone for each skin characteristic. A score of 5 is the greatest severity of the presence of the characteristic. And cumulative scores are derived as a code to indicate skin status at the time of evaluation. The evaluation is conducted with defined criteria to minimize inter-evaluator error.

The skin status quo code, intrinsic code and cosmetic code deriving methods and systems of this disclosure are merely exemplary embodiments and are not intended to limit the disclosure. The method and system of this disclosure represent a seamless cosmetic intervention cycle for an individual from a doctor or practitioner's practice room to the individual's daily life.

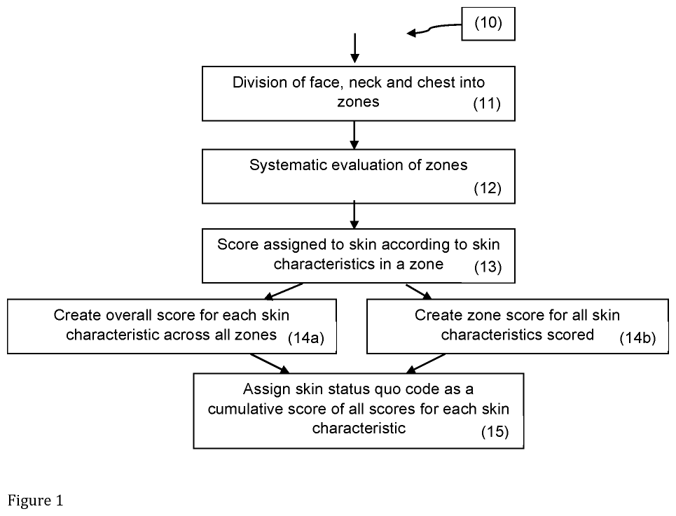

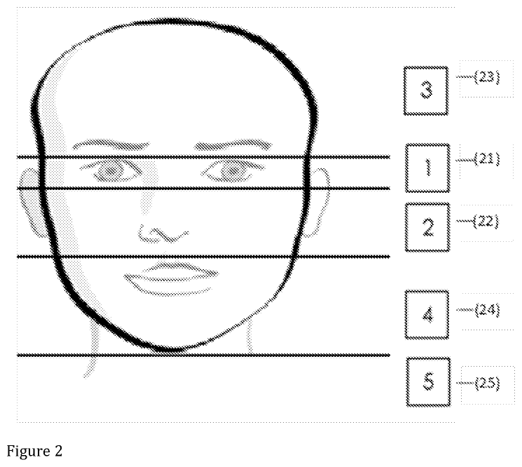

FIG. 1 is a diagram illustrating in general terms a method (10) of assigning a skin status quo code. First is the step (11) is the dividing the different areas of a person's skin such as face, neck and/or chest of a person into zones, the number of zones may vary from 1 to 15. A preferred example of the division of a face, neck and chest into zones (20) is, as in the diagrammatic representation of FIG. 2, wherein a person's face, neck and chest is divided into 5 zones. Zone 1 (21) is an area of the face that includes eyes and temple, zone 2 (22) is the area of the face that includes cheeks, nose and ears, zone 3 (23) is the area of the face that includes the forehead and eyebrows, zone 4 (24) is the area of the face that includes the person's moth, chin and jaw and zone 5 (25) is the neck and chest.

The step (12) of evaluation for skin characteristics in each zone is followed by a step (13) of assigning scores to each zone for the skin characteristics. This is followed by either steps (14a) or (14b). In Step (14a) the individual scores for each skin characteristic in each zone (21), (22), (23), (24) and (25) is added together to arrive at an overall score of a skin characteristic and in Step (14b) a zone score for each zone for the specific skin characteristic is created. Whereafter a skin status quo code is derived which is a cumulative score of the various skin characteristics evaluated for all zones evaluated and scored (step (15)).

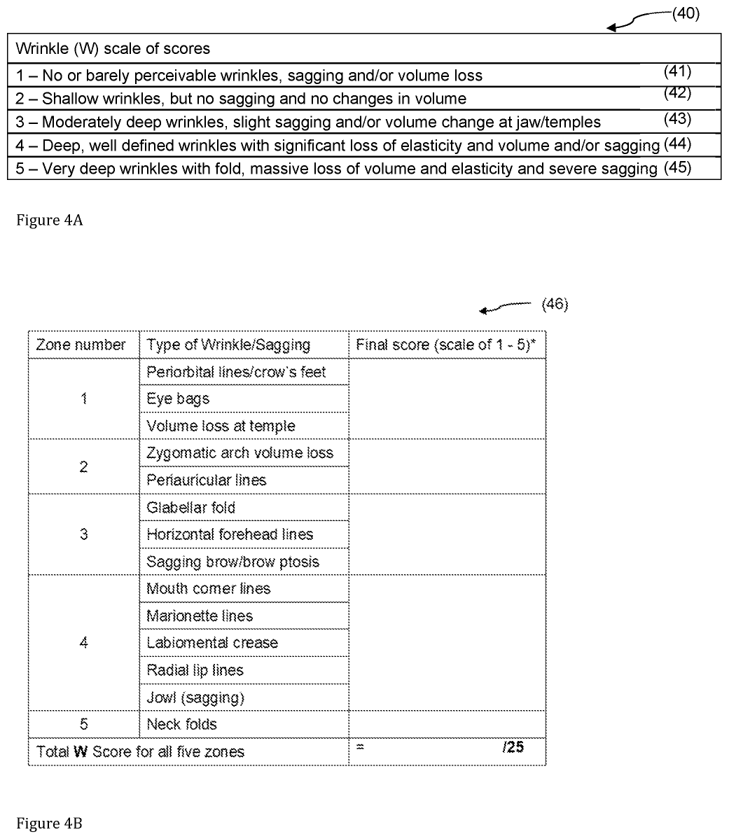

Example methods of evaluating the skin characteristics of wrinkles, sensitivity, pigmentation and dryness and assigning a score for each of the mentioned skin characteristics in each zone is as illustrated in FIGS. 4, 5, 6 and 7 respectively.

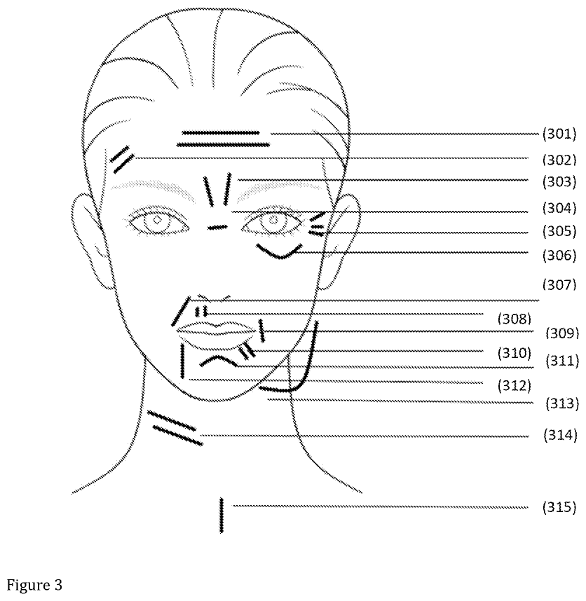

A preferred example of the evaluation criteria and scale of scores for the skin characteristic of wrinkle, (40) is as illustrated in FIG. 4A and a corresponding scoring template example is illustrated in FIG. 4B. In this disclosure a wrinkle scoring scale (40) is from 1 to 5. An evaluation criteria to arrive at a score of 1 (41) for a none or barely perceivable wrinkles, sagging and volume loss to a maximum score of 5 (45) for very deep wrinkles with fold, massive loss of volume, elastivcity and severe sagging, is as represented in FIG. 4A. FIG. 3 is a diagrammatic example of some of the various areas on the face, neck and chest (30) that are evaluated namely for wrinkles in the periorbital area (306), forehead (301), and mouth (309), crows' feet (305), bunny lines (304), marionette lines (312), vertical lip lines (308) (309) (310), frown/worry lines (301) and nasolabial fold (307) and also a loss of volume typically presents as hollow temples (302), sagging jowl (313), droopy eyebrows (303), eyebags at the tear trough (306), mental crease (301) and platysmal bands (315).

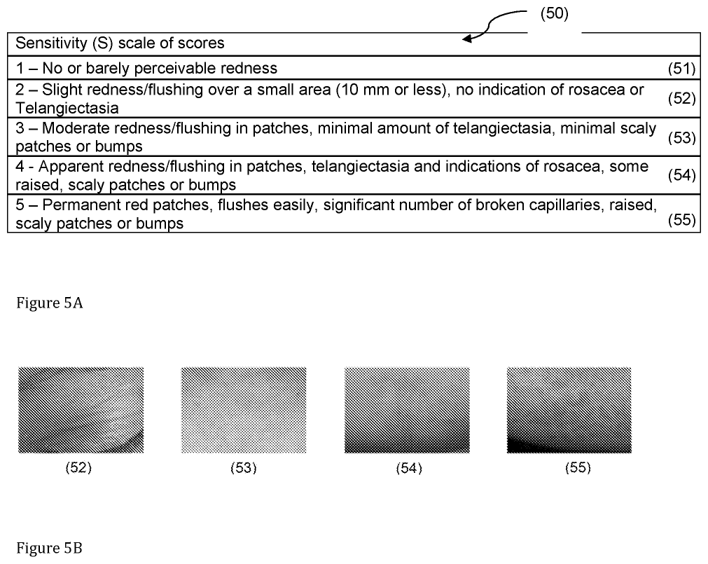

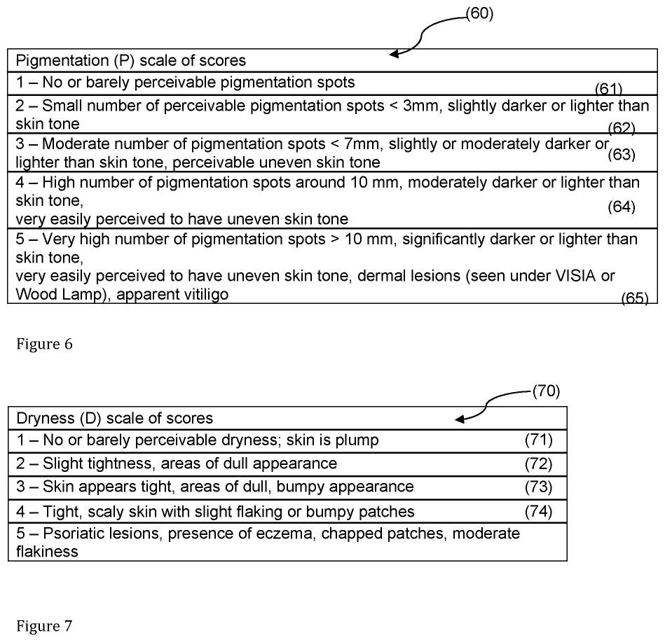

A preferred example of the evaluation criteria and scale of scores for the skin characteristic of sensitivity, corresponding to specific parameters (50) is illustrated in FIG. 5A. The presentation of visible inflammation and redness in the skin have associated scale of scores of 1 (51) to 5 (55). Wherein a score of 1 is none or barely perceivable redness, to a score of 5 being representative of permanent red patches, a skin that flushes easily, with significant number of broken capillaries, rasied scaly patches or bumps. A common area in which this is observed is the nose and cheeks in zone 2 (22). Telangiectasias or broken capillaries contribute towards a high scoring in this area. Flushed cheeks, scaling and fine bumps are common indicators of inherent sensitivity. In Fitzpatrick Type V and VI, this parameter would be described by taking an image using the VISIA™ system (or similar) to visualize inherent redness masked by melanin. Alternatively, it would be scored by unevenness of skin tone, not to be confused with the presence of pigmentation. FIG. 5B is a representative visual skin guide for the scoring, for scores 2 to 5 for sensitivity, and is denoted by reference (52) to (55), sensitivity visual guide for sensitivity for score 1 is an absence of redness and therefore not shown in the visual guide.

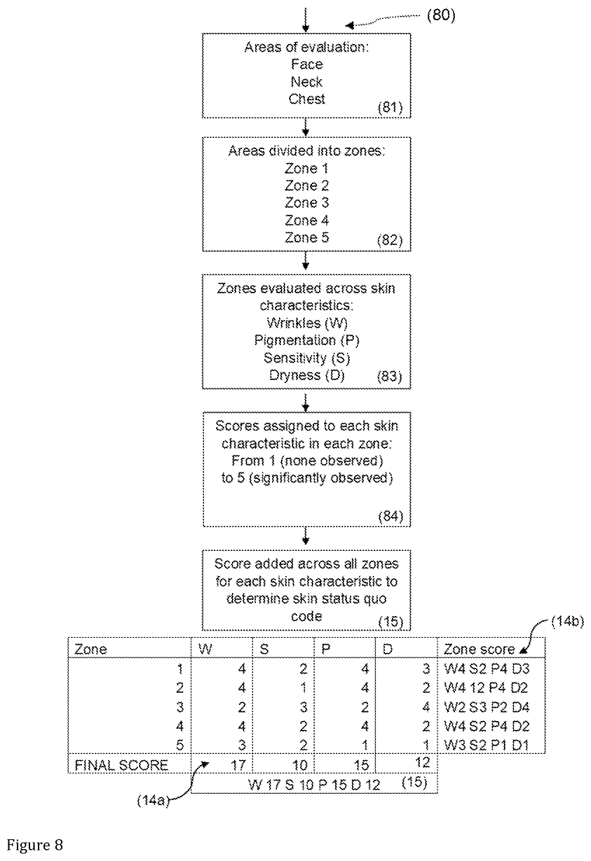

A preferred example of the evaluation criteria and scale of scores corresponding to specific parameters (60) for the skin characteristic of skin pigmentation is illustrated in FIG. 6. The evaluation of pigmentation mainly considers the general evenness of the skin's appearance, including considerations of both hyper- and hypopigmentation which maybe as a result of a variety of factors: aging; hormones; sun exposure; and inflammation (for example, acne or from irritants such as perfume) and often seen in the zonal areas (23), (25) and (21) and specifically seen on the forehead, upper lip and under eyes respectively in these zones. Extrinsic factor such as Solar lentigines also known as liver/age/brown/sun spots from light brown to black in colour, appear in areas of sun damage all over the body, often presenting above the age of 40 are also taken into consideration in this evaluation. Post-inflammatory hyperpigmentation: localized areas of hyperpigmentation from previous aesthetic treatments (laser/chemical peels), reaction to topical product, as a result of acneic lesions is also considered. In FIG. 6 from the scale of scores presented, it is clear that a score of 1 is when there are none or barely perceivable pigmentation (61), 3 being a score of moderate number of pigmentation spots <7 mm, slightly or moderately darker than the individual's skin tone with a perceivable uneven skin tone (63), to an extreme score of 5 being a very high number of pigmentation spots >10 mm, significantly darker than skin tone, very easily perceived to have an uneven skin tone (65), dermal lesions visible under VISIA™ or Wood Lamp. It is desirable that a Wood Lamp examination is conducted since unlike dermal and mixed lesions, epidermal lesions tend to be well circumscribed and are enhanced under the Wood Lamp. Dermal lesions carry a higher score than epidermal lesions in this method of scoring for pigmentation.

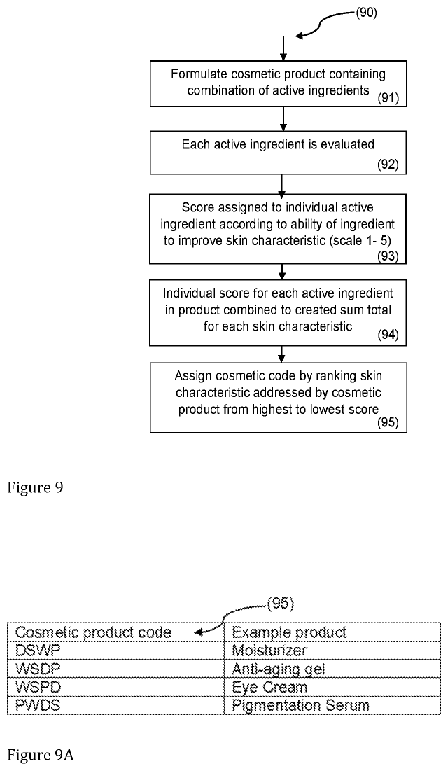

A preferred example of the evaluation criteria and scale of scores (70) for the skin characteristic of dryness is illustrated in FIG. 7. Dryness presents on a person's skin as flakiness, lack of radiance, tightness (loss of pliability) and rough texture and the score of 1—is indicative of none or barely perceivable dryness and the skin is plump (71), a score of 2 wherein slight tightness or areas of dull appearance are visible (72) to a score of 5 is typically associated with psoriatic lesions, presence of eczema, chapped patches and moderate flakiness (75). These parameters are seen more drastically on the cheeks and around the eye area as likely to result in the scores in zones 1 (22) and 4 (22).

These preferred embodiments of evaluation are examined within each zone and in a preferred embodiment a zonal score is (14b) created to identify zones that are specific concerns for skin treatment. It is a desirable object of this disclosure that cosmetic interventions are specific and directed to skin characteristics for each zone and not generalized.

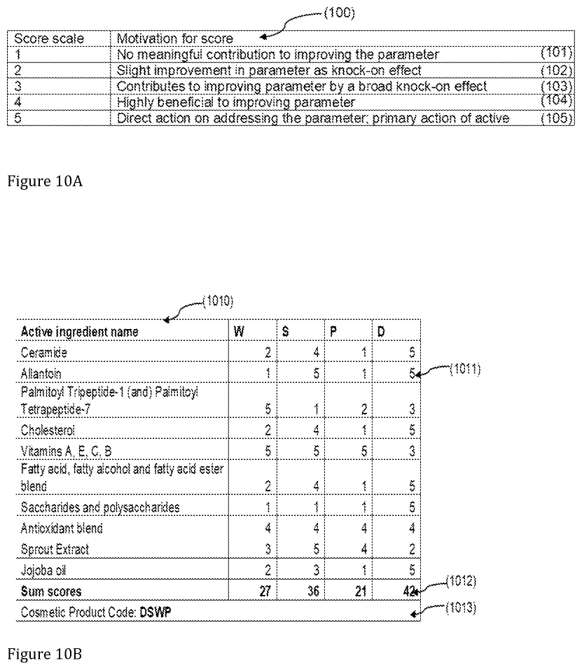

An exemplary embodiment of the method of assigning a skin status quo code as an easily understandable, empirical and practical representation of the current skin appearance and health based on skin characteristics of wrinkling, dryness, sensitivity and pigmentation is illustrated in FIG. 8. According to the preferred embodiment of the scoring method as cited above, the face, neck and chest (20) are divided into 5 zones (21), (22), (23), (24) and (25) respectively and each zone is evaluated for the four skin characteristics of wrinkles, sensitivity, pigmentation and dryness (82) scored systematically (83). The scoring for each zone on a scale of 1 to 5, may be a minimum score of 1 and a maximum score of 5, and the score for the skin characteristic as a combined total of the scores in each zone maybe a maximum of 25 and a minimum of 5. In the preferred embodiment each zone is analysed for four skin characteristics (Wrinkles, Sensitivity, Pigmentation and Dehydration, or WSPD) on a scale of 1-5, where 1 is where the skin characteristic is insignificantly affected and 5 is severely affected. Scores added up to arrive at the zone score code (14b) to identify characteristics of concern in the zone (83) or scores added up across the skin characteristics to arrive at the final score code and is the skin status quo code (15). Herein in the example illustrated in FIG. 8 the subject is denoted to have a current skin status code (15) at the time of evaluation as W₁₇S₁₀P₁₅D₁₂.

An analysis of the score is indicative of a subject who needs attention for the skin characteristics in the order of W>P>D and then S, such that a subject with high wrinkles and loss of firmness, as well as pigmentation.

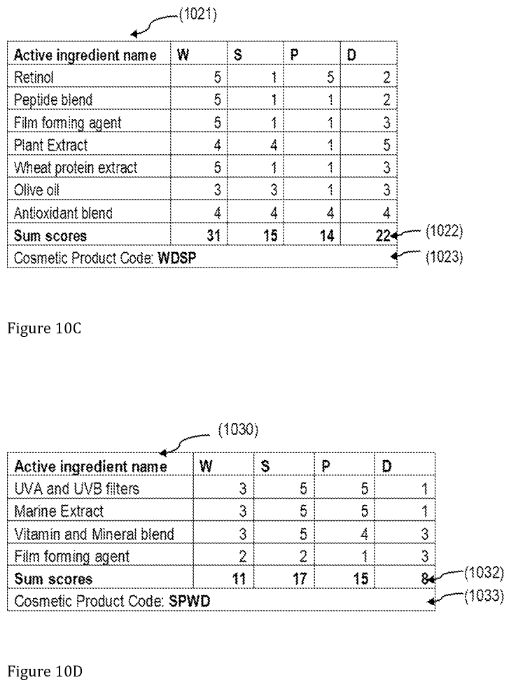

A preferred embodiment of scoring actives within a cosmetic product to arrive at a cosmetic code is illustrated by way of an example in FIG. 9. According to this example method (90), a cosmetic product as formulated has a combination of active ingredients (91), each active ingredient is evaluated (92), scores are assigned for the active ingredient based on its ability to effect specific skin characteristics (93) a sum of scores for each characteristic is calculated (94) and the cosmetic is then assigned a cosmetic code which is a code derived on the basis of ranking of the sum of scores for the skin characteristic (95). The cosmetic code in the preferred embodiment is a given four-letter code as a combination of W/P/D/S, where the characteristic category for which the cosmetic is effective is listed in descending order, from the characteristic having the highest scoring based on the sum of scores for actives in that category to the lowest.

A list of cosmetic codes and a typical example product is listed as an example of how a cosmetic product is assigned a cosmetic code in FIG. 9A. As illustrated in FIG. 9A a cosmetic code DSWP is indicative of a cosmetic likely to be most effective against the treatment of Dryness and in descending order of D>S>W>P and is typically indicative of a moisturizer.

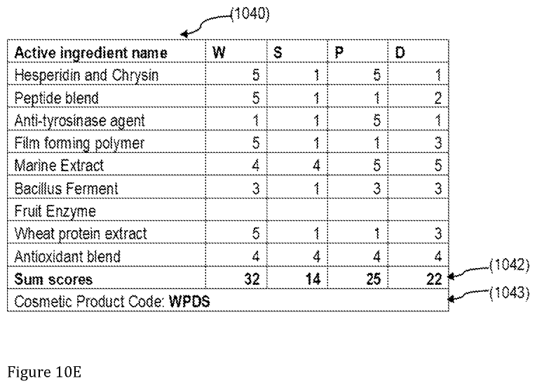

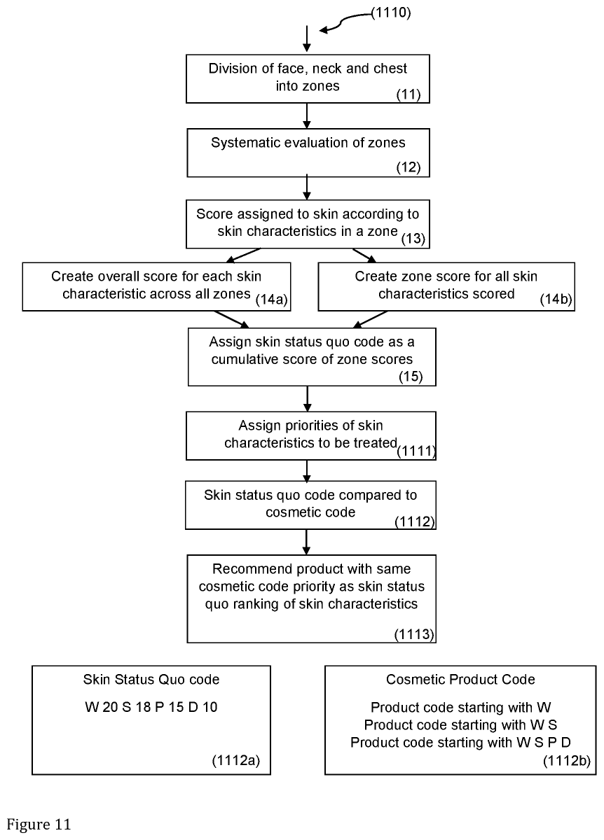

FIG. 10A is an example of the motivation of the scores and scale of scores from 1 to 5 (100) assigned to an active for a skin characteristic. In this example each active ingredient of a cosmetic is chosen (91), evaluated (92) and given a score between 1-5 (93) according to its ability to address the skin characteristics of W (wrinkles), P (pigmentation), D (dryness) and S (sensitivity). All scores associated with each active for a skin characteristic is from prior art studies for individual characteristics and benefits associated with the active for the skin characteristic. The preferred scoring technique in this embodiment is to assign a score of 1 representing that the active has no meaningful contribution to improving a skin characteristic (101), a score of 2 being a slight improvement in a skin characteristic attributable to the active (102), a score 3 of being the active contributes to a skin characteristic by a broad knock-on effect (103), score 4 for the active being representative that the active is highly beneficial to improving a skin characteristic (104) and a score 5 is assigned when the active has a direct and primary mode of action on a skin characteristic (105).

An example of an active for skin hydrating, calming properties is allantoin. The example of scoring of allantoin is illustrated using the scale of scores of FIG. 10A. Allantoin is assigned a score of 5 for the S or sensitivity skin characteristic. Allantoin is also known to boost skin barrier function, in turn typically having the ability to reduce amount of water lost from the skin of a person, therefore allantoin has a large impact on the D or dryness characteristic and would also have a score of 5 for the skin characteristic of D (105). The mechanisms of action for allantoin as per prior art, do not have a perceivable influence on the W or P characteristics, and both are therefore assigned a value of 1 respectively. Therefore allantoin would be assigned an active score for the skin characteristics of W, S, P and D as 1, 5, 1 and 5 respectively (1011) W₁S₅P₁D₅.

FIG. 10B is an illustrative example of the scoring of actives in a preferred embodiment of a cosmetic product chosen as a moisturizer. Herein the scoring of each of the actives is illustrated based on its ability to affect the characteristics of W, S, P and D. The cosmetic code of DSWP is arrived at as the summed up scores of actives; ceramide, allantoin, Palmitoyl Tripeptide-1 (and) Palmitoyl Tetrapeptide-7, cholesterol, Vitamins A, E, C and B, Fatty acid, fatty alcohol and fatty acid ester blend, saccharides and polysaccharides, antioxidant blend, sprout extract and jojoba oil for the characteristics of W, S, P and D. The scores are 27, 36, 21 and 42 for W, S, P and D respectively (1012). The scores when organized in a decreasing order of effect on the skin characteristic are 42>36>27>21 and therefore when the characteristics are aligned by the same ranking they are D>S>W>P, in this embodiment of a cosmetic the cosmetic is assigned a cosmetic product code of DSWP. Typically moisturizers have a cosmetic code of DSWP (1013).

An example of the actives typically included, but not limited to, in a mosituriser include aqua, ceramide, allantoin, sodium PCA, palmitoyl Tripeptide-1 (and) Palmitoyl Tetrapeptide-7, cholesterol, Vitamin A, Vitamin E, Vitamin C, Vitamin B, fatty acids, fatty alcohol, fatty acid esters, saccharide isomerate, hyaluronic acid, Lepidium Sativum (sprout) extract, Simmondsia chinensis (jojoba) seed oil, glycerin, phenoxyethanol, cetyl alcohol, stearic acid, lecithin, carbomer and xanthan gum, and a combination of any of the foregoing.

In an example of the preferred embodiment of an anti-aging firming gel in FIG. 100, the active ingredients in this composition are retinol, peptide blend, film forming agent, plant extract, wheat protein extract, olive oil and an antioxidant blend (1020). The scoring of each of these actives for the skin characteristics of W, S, D and P (100) reveal a final score of 31, 15, 14 and 22 respectively (1022). When the characteristics are organized in descending order of sum scores of the characteristics they follow that the cosmetic code thereof derived is WDSP (1023). Typically anti-aging firming gel formulations, exhibit an active scoring pattern that is characterized in descending order of sun scores for the characteristics as W>D>S>P and the cosmetic product code is WDSP.

An example of the actives typically included, in an anti-aging firming gel include, but not limited to, are aqua, Helianthus Annuus (Sunflower) Seed Oil, Butylene Glycol, Sodium Acrylates Copolymer, Glycerin, Hydrogenated Polyisobutene, Maltodextrin, Caesalpinia Spinosa Gum, Phospholipids, Polyglyceryl-10 Stearate, Phenoxyethanol, Glycosaminoglycans, Sodium PCA, Urea, Sodium Hydroxide, Glycogen, Trehalose, Disodium EDTA, Fragrance, Pentylene Glycol, Ethylhexylglycerin, Hydrolyzed Rice Protein, Hexylene Glycol, Polyquaternium-51, Tropaeolum Majus Flower/leaf/stem Extract, Caprylyl Glycol, Triacetin, Litchi Chinensis Pericarp Extract, Wine, Tocopherol, Sodium Hyaluronate, Coenochloris Signiensis Extract, Lecithin, capric/caprylic triglycerides, acrylates copolymer, carbomer, pullulan, retinol, retinol palmitate, retinol ester, peptide blend (peptide-4, peptide-7, peptide-33, oligopeptide), plant extract (preferably bioflavonoids or leontopodium alpinum extract), wheat protein extract, Olea Europaea (olive) oil, ascorbic acid, sodium ascorbyl phosphate, vitamin E, thioctic acid, camellia oleifera (green tea) leaf extract and hyaluronic acid, and a combination of any of the foregoing.

In an example of a preferred embodiment of a sun screen formulation, FIG. 10D, the actives UVA and UVB filters, marine extract, vitamin and mineral blend and film forming agent (1030) are evaluated for their effects on the skin characteristics of W, S, P and D (100) and scored. The formulation is assigned a final sum score pattern of W, S, P and D of 11, 17, 15 and 8 respectively (1032). The characteristics can be aligned according to the decreasing order of their respective scores as S>P>W>D, the cosmetic product code is SPWD (1033).

An example of actives typically included in a sunscreen formulation, but not limited to, are aqua, isostearyl isostearate, beeswax, pullulan, titanium dioxide, zinc oxide, octocrylene, vitamin E, vitamin C, vitamin B3, vitamin B5, polyurethane, phenoxyethanol, dimethicone, avobenzone and ethylhexyl methoxycinnamate, and a combination of any of the foregoing.

In an example of a preferred embodiment of a topical eye cream formulation FIG. 10E, the actives hesperidin and chrysin, peptide blend, anti-tyrosinase agent, film forming polymer, marine extract, bacillus ferment, fruit enzyme, wheat protein extract and antioxidant blend (1040) are evaluated for their effects on the skin characteristics of W, S, P and D (100) and scored. The formulation is s assigned a final sum score pattern of W, S, P and D of 32, 14, 25 and 22 respectively (1042). The characteristics can be aligned according to the decreasing order of their respective scores as W>P>D>S, the cosmetic product code is WPDS (1043).

An example of actives typically included, in a topical eye cream formulation are, but not limited to, Aqua, tetrapeptide-4, ellagic acid, algae extract, Helianthus Annuus (Sunflower) Seed Oil, Butylene Glycol, Sodium Acrylates Copolymer, Glycerin, Hydrogenated Polyisobutene, Maltodextrin, Phenoxyethanol, Phospholipids, Polyglyceryl-10 Stearate, Macrocystis Pyrifera Extract, Sodium PCA, Urea, Isohexadecane, Hydrolyzed Wheat Protein, PVP, Disodium EDTA, Fragrance, Sodium Hydroxide, Trehalose, PEG-40 Stearate, Pentylene Glycol, Ethylhexylglycerin, Hydrolyzed Rice Protein, Hexylene Glycol, Cetearyl Methicone, Steareth-2, Polyquaternium-51, Tropaeolum Majus Flower/leaf/stem Extract, Caprylyl Glycol, Triacetin, Litchi Chinensis Pericarp Extract, Tocopherol, Sodium Hyaluronate, Coenochloris Signiensis Extract, Lecithin, and a combination of any of the foregoing.

Suitable actives include, but are not limited to, anti-acne agents, antimicrobial agents, anti-inflammatory agents, analgesics, antietythemal agents, antipruritic agents, antiedemal agents, antipsoriatic agents, antifungal agents, skin protectants, Sunscreen agents, Vitamins, antioxidants, Scavengers, antiirritants, antibacterial agents, antiviral agents, antiaging agents, protoprotection agents, hair growthenhancers, hair growth inhibitors, hair removal agents, antidandruff agents, anti-Seborrheic agents, exfoliating agents, wound healing agents, anti-ectoparacitic agents, anti-pigmentation agents, whiteners, moisturizing agents, Sebum modulators, immunomodulators, hormones, botanicals, moisturizers, astringents, cleansers, Sensates, antibiotics, anesthetics, Steroids, tissue healing Substances, tissue regenerating Substances, amino acids, peptides, minerals, ceramides, biohyaluronic acids, and any combination of any of the foregoing.

Preferred anti-acne agents include, but are not limited to, Salicylic acid, retinoic acid, alpha hydroxy acid, benzyl peroxide, Sodium Sulfacetamide, clindamycin, and any combination of any of the foregoing.

Suitable antimicrobial agents include, but are not limited to, benzalkonium chloride, benzethonium chloride, chlorhexidine gluconate, chloroxylenol, cloflucarban, fluorosalan, hexachlorophene, hexylresorcinol, iodine complex, iodine tincture, para chloromercuriphenol, phe nylmercuric nitrate, thimerosal, vitromersol, Zyloxin, triclocarban, triclosan, methyl-benzethonium chloride, nonyl phenoxypoly(ethyleneoxy) ethanol-iodine, para chloro-meta-Xylenol, providone-iodine complex, poloxamer-iodine complex, triclocarban, undecoylium chloride-iodine complex, phenoxyethanol, and any combination of any of the foregoing.

Suitable antiinflammatory agents include, but are not limited to, alidoxa, allantoin, aloe Vera, aluminum acetate, aluminum hydroxide, bismuth Subnitrate, boric acid, calamine, casein, cellulose, microporous, cholecatciferol, cocoa butter, cod liver oil, colloidal oatmeal, cysteine hydrochloride, dexpanthenol, dimethicone, glycerin, kaolin, lanolin, live yeast cell derivative, mineral oil, Peruvian balsam, petrolatum, protein hydrolysate, racemethionine, Shark liver oil, Sodium bicarbonate, Sulfur, talc, tannic acid, topical Starch, Vitamin A, Vitamin E, white petrolatum, Zinc acetate, Zinc carbonate, Zinc oxide, hydrocortisone, betamethasone, ibuprofen, indomethicin, salicylic acid, acetyl salicylic acid, tacrolimus, flucoinolone acetonide, Sodium Sulfacetamide, and any combination of any of the foregoing.

Suitable analgesics include, but are not limited to, diphenhydramine, tripeiennamine, benzocaine, dibucaine, lidocaine, tetracaine, camphor, menthol, phenol, resorcinol, matacresol, juniper tar, methylsalicylate, turpentine oil, capsicum, methyl nicotinate, b-glucan, and any combination of any of the foregoing.

Suitable antietythermal agents include, but is not limited to, tetrahydrozoline and hydracortisone.

Suitable antipruritic agents include, but are not limited to, benadryl, pramoxine, antihistamines, and any combination of any of the foregoing.

Suitable antiedemal agents, include, but are not limited to, pregnenalone acetate, taninglyrosides, and any combination of any of the foregoing.

Suitable antipsoriatic agents include, but are not limited to, caleipotriene, coal tar, anthralin, Vitamin A, and any combination of any of the foregoing. Preferred combinations of antipsoriatic agents include, but are not limited to, hydrocortisone, retinoic acid, and alpha hydroxy acid; do vone X, Salicylic acid, and a Sunscreen agent; indomethicin, Salicylic acid, and urea; anthralin and Salicylic acid; and anthralin and indomethicin. Other Suitable antipsoriatic agents include, but are not limited to, caleipotriene, coal tar, anthralin, Vitamin A, and any combination of any of the foregoing.

Suitable antifungal agents include, but are not limited to, clioquinol, haloprogin, miconazole nitrate, clotrimazole, metronidazole, toinaftate, undecylenic acid, iodoquinol, and any combination of any of the foregoing.

Suitable skin protectants include, but are not limited to, cocoa butter, dimethicone, petrolatum, white petrolatum, glycerin, Shark liver oil, allantoin, cholesterol, phospholipids, polyurethane and any combination of any of the foregoing.

Suitable Sunscreen agents include, but are not limited to, ethylhexyl methoxycinnamate, avobenzone, benzophenone 3, octoacrylene, titanium dioxide, diethylamino hydroxybenzoyl hexylbenzoate, Zinc oxide, and any combination of any of the foregoing.

Suitable antioxidants include, but are not limited to, Scavengers for lipid free radicals and peroxyl radicals, quenching agents, and any combination of any of the fore going. Suitable antioxidants include, but are not limited to, tocopherol, BHT, beta carotene, Vitamin A, ascorbic acid, ubiquinol, ferulic acid, azelaic acid, thymol, catechin, Sinapic acid, EDTA, lactoferrin, roSmariquinone, hydroxytyroSole, Sesamol, 2-thioxanthine, nausin, malvin, carvacone, chalcones, glutathione isopropyl ester, Xanthine, melanin, guanisone, lophorphyrins, 8-hydroxy Xanthine, 2-thioxanthione, Vitamin B2, plant alkaloids, catalase, quercetin, tyrosine, SOD, cysteine, methionine, genistein, NDG, procyanidin, hamamelitannin, ubiquinone, trolox, licorice extract, propyl gallate, Sinapic acid, and any combination of any of the foregoing. Suitable vitamins include, but are not limited to, Vitamin E, Vitamin A palmitate, Vitamin D, Vitamin F, vitamin B, Vitamin B, Vitamin B, Vitamin C, ascorbyl palmitate, Vitamin E acetate, biotin, niacin, DL-panthenol, and any combination of any of the foregoing.

Suitable amino acids include, but are not limited to, glycine, Serine, and any combination of any of the foregoing.

Suitable Vitamin combinations include, but are not limited to, Vitamins A, E, C, B and, typically may include Vitamin A or a combination or any of its derivatives including retinol, retinaldehyde, retinoic acid, retinol, retinyl palmitate, retinol esters, Vitamin E typically may include Vitamin E or a combination or any of its derivatives including alpha-tocopherol, gamma-tocopherol, tocopheryl acetate, tocotrienol, Vitamin C typically may include Vitamin C or a combination of or any of its derivatives including ascorbic acid, magnesium ascorbyl phosphate, ascorbyl palmitate, ascorbic acid polypeptide, ascorbyl glucosamine, ascorbyl glucoside, Ester-C, sodium ascorbyl palmitate, sodium ascorbyl phosphate, vitamin B typically may include Vitamin B or a combination or any of its derivatives including thiamin (B1), riboflavin, niacin, niacinamide, pantothenic acid, biotin, vitamin B6 (pyridoxine), folate, folic acid, vitamin B12 (cyanocobalamin), and any combination of any of the foregoing.

Fatty acid, fatty alcohol and fatty acid ester blend typically include, but are not limited to, stearic acid, caprylic acid, oleic acid, myristic acid, palmitic acid, lauric acid, or linoleic acid and alpha-linolenic acid, cetyl alcohol, lauryl alcohol, cetostearyl alcohol, stearyl alcohol, oleyl alcohol, isopropyl myristate, isopropyl palmitate or glyceryl stearate, and any combination of any of the foregoing.

Saccharides and polysaccharides typically include, but are not limited to, saccharide isomerate, xylitol, dextrose, agar, alginic acid, carrageen, CM-Glucan, chitin, dextran, carboxy methyl cellulose, glycogen, guar gum, hydroxypropyl starch phosphate, gum Arabic, hyaluronic acid, hydroxyethyl cellulose, methyl cellulose, mucopolysaccharides (glycosaminoglycans), pectin, alkyl polyglycosides, xanthan gum, mannose or glucuronic acid, and any combination of any of the foregoing.

Antioxidant blend typically include, but are not limited to, vitamin A or its derivatives retinal, retinaldehyde, retinoic acid, retinol, retinyl palmitate, retinol esters, vitamin E or any of its derivatives including alpha-tocopherol, gamma-tocopherol, tocopheryl acetate, tocotrienol, Vitamin C or any of its derivatives including ascorbic acid, magnesium ascorbyl phosphate, ascorbyl palmitate, ascorbic acid polypeptide, ascorbyl glucosamine, ascorbyl glucoside, Ester-C, sodium ascorbyl palmitate, sodium ascorbyl phosphate including other compounds such as tripeptide-33, lycopene, ubiquinone, idebenone, Camellia sinensis, silymarin, milk thistle extract, Coffea arabica extract, resveratrol, Vitis vinifera extract, Punica granatum extract, genistein, pycnogenol, niacinamide, Carthamus tinctorus extract, beta-carotene, omega-3 fatty acids, omega-6 fatty acids, carthamus tinctorus seed oil, tagetes erecta flower extract or thioctic acid, and any combination of any of the foregoing.

Sprout Extract typically include, but are not limited to, broccoli sprout extract, garden cress sprout extract (Lepidium sativum extract), spinach sprout extract, mustard sprout extract, sunflower sprout extract, sulforaphane, pea sprout extract, wheat sprout extract (Triticum vulgare extract), and black soybean sprout extract, and any combination of any of the foregoing.

Peptide blend typically include, but are not limited to, peptides suitable for topical application, or as disclosed in Schagen, S. K. (2017) entitled: Topical peptide treatments with effective anti-aging results. Cosmetics, 4(2), 16, and any combination of any of the foregoing.

Film forming agent typically include, but are not limited to, compounds of a polymeric structure that creates a film on the skin, including sodium hyaluronate, Polyurethane-36, -14, -28, -42, -60, -61, -62 and -69, carboxy-methylcellulose, Kappaphycus alvarezii Extract, Caesalpinia spinosa Fruit Extract, pullulan, biopolymer BHA-10, dioctyl malate, dioctyl maleate, vegetal DNA, phospholipids, ethylene acrylate copolymer, acrylates copolymer, or acrylamide copolymer, and any combination of any of the foregoing.

Plant Extract typically include, but are not limited to, extracts from a plant material as disclosed in Aburjai, T., & Natsheh, F. M. (2003) entitled: Plants used in cosmetics. Phytotherapy research, 17(9), 987-1000), Zhu, W., & Gao, J. (2008) entitled: The use of botanical extracts as topical skin-lightening agents for the improvement of skin pigmentation disorders. Journal of Investigative Dermatology Symposium Proceedings, 13, No. 1, pp. 20-24, Elsevier, Ribeiro, A. S., et al (2015) entitled: Main benefits and applicability of plant extracts in skin care products. Cosmetics, 2(2), 48-65, Mukherjee, P. K., et al (2011) entitled: Bioactive compounds from natural resources against skin aging. Phytomedicine, 19(1), 64-73), or Allemann, I. B., & Baumann, L. (2009) entitled: Botanicals in skin care products. International journal of dermatology, 48(9), 923-934), and any combination of any of the foregoing.

Wheat protein extract typically include, but are not limited to, a hydrolyzed wheat protein, hydrolysed wheat gluten, or wheat germ oil, and any combination of any of the foregoing.

Marine extract typically include, but are not limited to, seaweed, microalgae, sea algae and sea water extract, more specifically red (pyrphora) algae extract, black algae extract, giant kelp extract, brown (laminaria or sargassum) algae extract, snow algae extract, green (ecklonia) algae extract, phaeophycae laminariales extract, bladderwrack extract and those disclosed in Ariede, M. B., et al (2017) entitled: Cosmetic attributes of algae-A review. Algal Research, 25, 483-487) or marls sal, and any combination of any of the foregoing.

Vitamin and Mineral blend typically include, but are not limited to, vitamin A or any of its derivatives including retinal, retinaldehyde, retinoic acid, retinol, retinyl palmitate, retinol esters, Vitamin E or any of its derivatives including alpha-tocopherol, gamma-tocopherol, tocopheryl acetate, tocotrienol, Vitamin C or any of its derivatives including ascorbic acid, magnesium ascorbyl phosphate, ascorbyl palmitate, ascorbic acid polypeptide, ascorbyl glucosamine, ascorbyl glucoside, Ester-C, sodium ascorbyl palmitate, sodium ascorbyl phosphate, or Vitamin B or any of its derivatives including thiamin (B1), riboflavin, niacin, niacinamide, pantothenic acid, biotin, vitamin B6 (pyridoxine), folate, folic acid, vitamin B12 (cyanocobalamin), Vitamin K, Vitamin K oxide, Vitamin D or any of its derivatives, choline, sodium, calcium, magnesium, potassium, chlorine, sulfur, phosphorus, iodine, iron, zinc, copper, selenium, molybdenum, fluoride, manganese, boron, nickel and cobalt, and any combination of any of the foregoing.

UVA and UVB filters typically include, but are not limited to, Bemotrizinol, Bis-Ethylhexyloxyphenol Methoxyphenyl Triazine, Butyl Methoxydibenzoylmethane, Diethylamino Hydroxybenzoyl Hexyl Benzoate, Disodium Phenyl Dibenzimidazole Tetrasulfonate, Drometrizole TriSiloxane, Menthyl Anthranilate, Methylene Bis-Benzotriazolyl Tetramethylbutylphenol (nano), Terephthalylidene Dicamphor Sulfonic Acid, Zinc Oxide (nano and non-nano), 4-Methylbenzylidene Camphor, Benzophenone-3, Benzophenone-4, Diethylhexyl Butamido Triazone, Ethylhexyl Methoxycinnamate, Ethylhexyl Salicylate, Ethylhexyl Triazone, Ethylhexyl dimethyl PABA, Homomenthyl Salicylate, Isoamyl p-Methoxycinnamate, Octocrylene, PEG-25 PABA, Phenylbenzimidazol Sulfonic Acid, Polysilicone-15, or Titanium Dioxide (nano and non-nano), and any combination of any of the foregoing.

Anti-tyrosinase agent typically include, but are not limited to, tetrapeptide-PKEK, black rice oligopeptides, rice bran protein, or those disclosed by Chang, T. S. (2009) entitled: An updated review of tyrosinase inhibitors. International journal of molecular sciences, 10(6), 2440-2475), or disclosed in Pillaiyar, T., Manickam, M., & Namasivayam, V. (2017) entitled: Skin whitening agents: Medicinal chemistry perspective of tyrosinase inhibitors. Journal of enzyme inhibition and medicinal chemistry, 32(1), 403-425, and any combination of any of the foregoing.

Fruit Enzymes typically include, but are not limited to, bromelain (pineapple extract), papain (papaya extract), blueberry extract and Cucurbita Pepo (pumpkin extract), kiwi fruit extract or watermelon fruit extract, and any combination of any of the foregoing.

These are only a select group of possible actives in any cosmetic formulation. The total palette of actives is vast and is covered in relevant literature, Trade Journals such as the International Federation of the Society of Cosmetic Chemists, and texts such as the Handbook of Cosmetic Science and Technology (2001), the International Cosmetic Ingredient Dictionary and Handbook (8th edition), A consumer's dictionary of cosmetic ingredients (7th edition), MILADY skincare and cosmetic ingredients dictionary (4th edition) and the cosmetic bench reference are incorporated herein by reference.

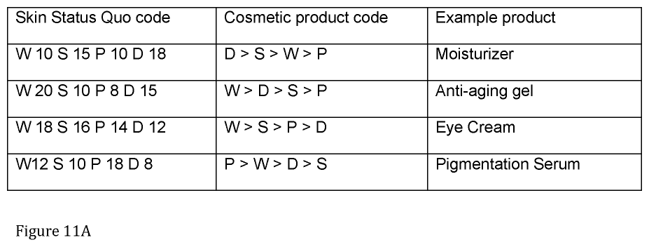

An exemplary embodiment of a method of recommending a cosmetic product with a cosmetic code is illustrated in FIG. 11. The method (1110) illustrates the division of the skin areas into zones (11), evaluating the zones for specific skin characteristics (12), scoring of the zones (13), either utilising one of two methods of cumulating the scores in (14a) and (14b), assigning a skin status quo code (15). Once a skin status quo code has been obtained, it is necessary to rank the skin priorities of characteristics that are to be treated (1111). Then compare (1112) the skin status quo code (15) based on the priority characteristics (1111) to a cosmetic code. Assign a cosmetic product with a cosmetic code based on priority of skin characteristics to be treated (1114). An illustration of assigning a cosmetic code based cosmetic product is when a person's skin status quo code is W20 S18 P15 D 10 (1114a), the order of priority of skin characteristics to be treated would be W>S>P>D and accordingly a cosmetic product with a cosmetic code of WSPD is assigned (1114b). FIG. 11A illustrates how the skin status quo code and priority rankings of the skin status quo code are aligned to a cosmetic product with a similar code. In another example if a subject has undergone the scoring method and has been told they have a score of W20 D18 P15 S8, the products that would be recommended would be products having a cosmetic code starting with a W or D. Typically the subject would be recommended the anti-aging firming gel and a topical eye cream.

Since a person's skin status quo code is a combination of the intrinsic (genetic) and extrinsic or external factors it follows that both these factors affect skin's characteristics. It is an embodiment of this disclosure to determine to what extent extrinsic/external factors contribute to a person's skin characteristics by comparing an individual's intrinsic score with the current skin status quo code.

Various SNPs are associated with its impact on multiple skin characteristics (Table 1). Table 1 is merely a sample of various SNPs and its impact on skin characteristics. It is envisaged that multiple other SNPs associated with its impact on skin characteristics can be utilised for a method of arriving at an intrinsic code for the predetermined skin characteristics of this disclosure or for, various other skin characteristics.

An embodiment of this disclosure accordingly relates to a method of assigning an intrinsic code for a subject. It is assumed that the genetics of an individual and their likelihood of changing their inherent characteristics in their lifetime, without interventions, is minimal. Therefore this disclosure associates the intrinsic code as a static code for an individual.

A desirable outcome of this disclosure is a method of determining an intrinsic code and a method to address skin characteristics, which are affected by external factors by comparing the current skin status quo code and intrinsic code. In this disclosure it follows that both the intrinsic and external factors would actively be addressed, as opposed to the prior art method of treating the symptomatic effects or trying to address inherent genetically related presentations in a subject, this embodiment of this disclosure allows a practitioner to address cosmetic interventions with a holistic understanding of the contributions of both the intrinsic and external factors.

TABLE 1
An example of SNPs and its association for its impact on skin
characteristics

| Gene | rs number | SNP | Skin category | Impact on skin |
|---|---|---|---|---|
| Matrix | rs1799750 | Insertion: 1G/2G | Firmness and | Variant associated |
| Metalloproteinase-1 | | | elasticity | with increased |
| (MMP1) | | | | collagen |
| | | | | breakdown |
| Collagen type I | various | various | Firmness and | Variations can |
| alpha I (COL1A1) | | | elasticity | lead to decreased |
| | | | | collagen |
| | | | | production |
| Melanocortin 1 | rs1805007 | Arg151Cys/R151C | Sun sensitivity | Variation |
| Receptor (MC1R) | | (C>T) | and | associated with |
| | | | pigmentation | greater |
| | | | | pheomelanin |
| | | | | production and |
| | | | | associated with |
| | | | | red hair and |
| | | | | freckles phenotype |
| Agouti Signalling | rs4911414 | G>T | Sun sensitivity | Tightly associated |
| Protein (ASIP) | | | and | with an increased |
| | | | pigmentation | likelihood of sun |
| | | | | sensitivity i.e. |
| | | | | sunburn and |
| | | | | freckles |
| Agouti Signalling | rs1015362 | 8818 G>A | Sun sensitivity | Tightly associated |
| Protein (ASIP) | | | and | with an increased |
| | | | pigmentation | likelihood of sun |
| | | | | sensitivity i.e. |
| | | | | sunburn and |
| | | | | freckles |
| X-Ray Repair | rs25489 | Arg280His/R280H | Sun damage, | The variation is |
| Cross- | | (G>A) | protection and | associated with |
| Complementing | | | repair | improved activity |
| Protein 1 (XRCC1) | | | | for conferring |
| | | | | protection against |
| | | | | DNA damage |
| | | | | caused by UV |
| | | | | exposure |
| X-Ray Repair | rs25487 | Q299R | Sun damage, | The variation is |
| Cross- | | | protection and | associated with |
| Complementing | | | repair | improved activity |
| Protein 1 (XRCC1) | | | | for conferring |
| | | | | protection against |
| | | | | DNA damage |
| | | | | caused by UV |
| | | | | exposure |
| 8-oxoguanine DNA | rs1052133 | Ser326Cys | Sun damage, | Variant associated |
| glycosylase 1 | | (C>G) | protection and | with decreased |
| (hOGG1) | | | repair | ability to repair |
| | | | | damaged DNA |
| Telomerase | rs401681 | C>T | Sun damage, | Variant (C allele) is |
| Reverse | | | protection and | associated with |
| Transcriptase | | | repair | decreased |
| (TERT) | | | | telomere length |
| | | | | and increased risk |
| | | | | of non-melanoma |
| | | | | skin cancers. |
| Superoxide | r54880 | C>T | Protection | Variation may lead |
| dismutase (SOD2) | | | from oxidative | to a change in |
| | | | stress | enzyme activity, |
| | | | | altering redox |
| | | | | balance. |
| Glutathione | rs1050450 | C>T | Protection | Variant is |
| peroxidase 1 | | | from oxidative | associated with |
| (GPx1) | | | stress | decreased enzyme |
| | | | | activity and |
| | | | | increased risk for |
| | | | | oxidative stress |
| Catalase (CAT) | rs1001179 | −262 C>T | Protection | Variant is |
| | | | from oxidative | associated with |
| | | | stress | decrease CAT |
| | | | | activity potentially |
| | | | | leading to |
| | | | | increased |
| | | | | oxidative stress. |
| NADP(H:) quinone | rs1800566 | Pro187Ser/C609T | Detoxification | Variant is |
| oxidoreductase 1 | | (C>T) | and skin | associated with |
| (NQO1) | | | sensitivity | decreased enzyme |
| | | | | activity and |
| | | | | increased risk for |
| | | | | oxidative stress |
| Glutathione S- | rs1695 | A>G | Detoxification | Variant is |
| Transferase P1 | | | and skin | associated with |
| (GSTP1) | | | sensitivity | decreased enzyme |
| | | | | activity and |
| | | | | increased risk of |
| | | | | skin sensitivities |
| Epoxide | rs1051740 | T>C | Detoxification | Variant has been |
| Hydroxylase | | | and skin | associated with |
| (EPHX1) | | | sensitivity | decreased enzyme |
| | | | | activity leading to |
| | | | | increased |
| | | | | oxidative stress, |
| | | | | skin sensitivity and |
| | | | | premature aging. |
| Interleukin 6 (IL6) | rs1800795 | G>C | Inflammation | The variant leads |
| | | | | to increased |
| | | | | expression with |
| | | | | higher baseline |
| | | | | levels of CRP |
| Interleukin 6 | rs2228145 | A>C | Inflammation | Variant has been |
| Receptor (1L6-R) | | | | associated to an |
| | | | | increased risk for |
| | | | | chronic low-grade |
| | | | | inflammation |
| C-Reactive Protein | rs1205 | G>A | Inflammation | Variant leads to |
| (CRP) | | | | higher levels of |
| | | | | CRP and risk for |
| | | | | chronic low-grade |
| | | | | inflammation |
| Tumour Necrosis | rs1800629 | G>A | Inflammation | Variation leads to |
| Factor Alpha | | | | increased risk for |
| (TNFA) | | | | skin sensitivity and |
| | | | | chronic low-grade |
| | | | | inflammation |

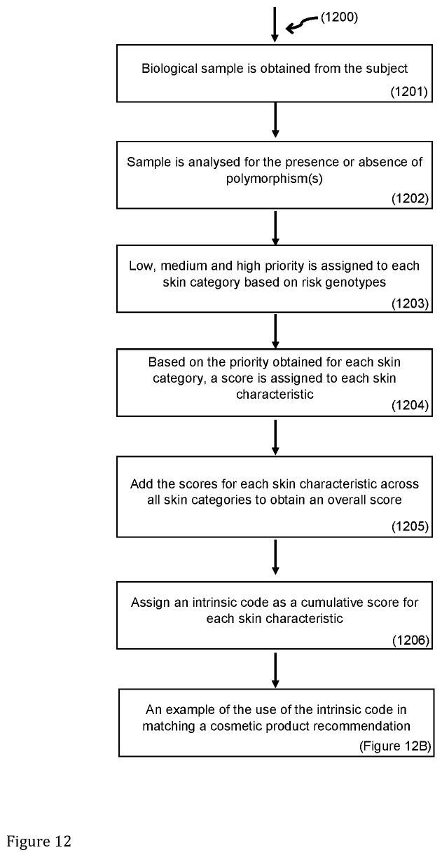

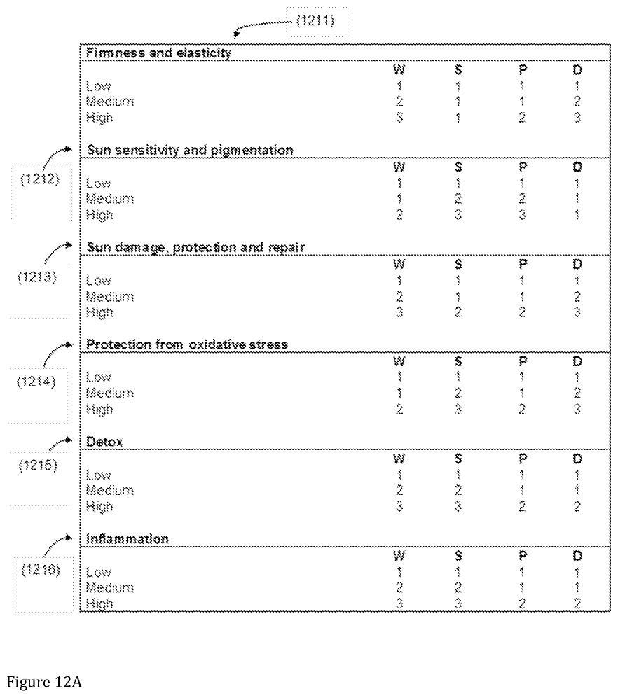

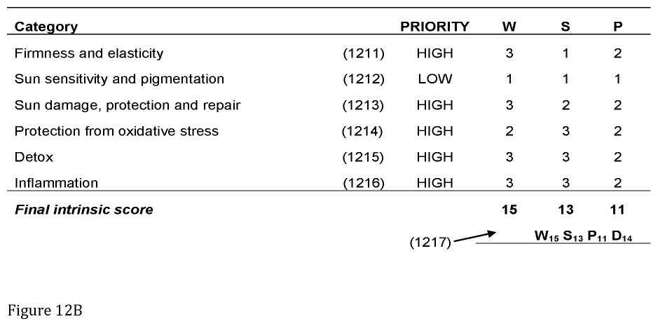

An example of a method of assigning an intrinsic score is illustrated in general terms in FIG. 12. As per the method (1200) a biological sample is obtained from a subject (1201), which may be a buccal swab or saliva or hair or blood sample. In an example of the method of analysis in this disclosure, the biological sample is sent to a commercial laboratory for isolation of the DNA and to obtain results of the SNP profiling, which include the presense or absence of SNPs (1202). The detection of polymorphism(s) as a tool in this present disclosure includes isolation, testing for polymorphisms and analysis by DNAlysis Biotechnology (https://dnalysis.co.za/) (“DNAlysis”). There is a risk associated with each genotype, so priority is assigned based on the polymorphism detected and the risk associated with that polymorphism. DNAlysis assigns the different skin categories a priority (low, medium and high) based on the presence or absence of polymorphisms based on the risks associated to the genotype (1204).

As per the example of scoring of low, medium or high priority risk of genotypes, in FIG. 12A, a numerical value is assigned to each skin characteristic according to the for each skin characteristic according to the priority ranking for each skin category.

FIG. 12 illustrates the scoring pattern wherein a high priority indicates that variations of polymorphism(s) detected in the genes represent high-risk genotypes with a strong association to a specific negative skin characteristic and assigned a score of 3; correspondingly medium priority represents a lower composition of risk genotypes and a score of 2 and a low priority indicates that little/no variations have been detected and for the skin characteristic the risks are the lowest and is assigned a score of 1.

The scoring pattern for a subject A who is a tennis coach, who eats well and exercises often is illustrated in FIG. 12B. DNAlysis assigned a priority level to each skin category after obtaining the biological sample and conducting the profiling for the detection of these polymorphisms (1202). In this example, a high priority was obtained for all categories except for sun sensitivity and pigmentation. Using the scoring method illustrated in FIG. 12A, the priority level assigned for each category is correlate to the score for each skin characteristic. For example, firmness and elasticity had a high priority and was assigned a score of 3 for W and D, a score of 2 for P and 1 for S. This scoring is conducted for all 6 skin categories of this embodiment, based on the priority levels assigned by DNAlysis. The overall scores for each skin characteristics are the added up to get a cumulative score of the scores for each skin category and skin characteristic which is, W15 S13 P11 D14 and is the intrinsic score for Subject A

Subject A, of the example of FIG. 12B, was scored by the method illustrated in FIG. 1, to arrive at a skin status quo code of W20 S6 P18 D17. Prior art teaches that the skin profile of a person is affected by a combination of extrinsic/external and intrinsic/inherent factors. A difference between a state of somebody's skin at the time of assessment such that their skin profile and genetic profile would be external factor influences and their contribution to skin characteristics. In calculating the difference between the skin status quo code and the intrinsic code, we arrive at a measure of the influence of external factors on the skin profile, based on specific skin characteristics and therefore the influence of the extrinsic factors is directed to those specific skin characteristics. The difference between the scores is W5 S−7 P7 D3 and this disclosure reveals that external factors influences W by 5, S by −7, D by 3 and P by 7. Based on the comparison between what is currently seen on the skin and the subject's genetic disposition as measured by the intrinsic code towards specific skin characteristics, it can be deduced that the extrinsic factors are impacting the appearance of pigmentation, wrinkles, followed by dehydration.

In this example, the external factor contribution to pigmentation is having the largest impact on the client's appearance, followed by both wrinkles and dehydration. As per the status quo code, these skin characteristics are of the greatest concern; they score highest and can inform an aesthetic practitioner's recommendation influence. In this example of this disclosure, the main priority for cosmetic interventions remains addressing wrinkles (status quo W score of 20), followed by pigmentation concerns (status quo P score of 18), and then dehydration and sensitivity. Wrinkles are the biggest concern and the pigmentation concern is due to the influence of external factors and the aesthetic practitioner's recommendation would be product with cosmetic code starting at W, with a supplementary recommendation of a product that addresses P.

An informed understanding of how the inherent code and extrinsic factors can not be utilised by the aesthetic practitioner and may recommend specific cosmetic interventions such as Botulinum toxin (to reduce lines associated with frowning in the harsh sun), micro needling with platelet rich plasma to address both wrinkling and pigmentation or laser to remove pigmentation spots.

Chemical peels may not be an appropriate recommendation since the subject, as a tennis coach who works outdoors, is exposed to UV rays from the sun. Herein, the practitioner or physician is able to recommend specific cosmetic products with a code PWDS and to further augment the cosmetic intervention for example an eye gel product with cosmetic code WSPD would be recommended, based on the high W score to compliment invasive or non-invasive cosmetic interventions already recommended.

In the example of Subject A herein, the skin status quo code assists the practitioner to make a recommended treatment plan, and provides a framework from which to reproducibly treat the skin and monitor progress and to subsequently treating the skin using the derived skin status quo code. A desirable object of this disclosure is the improvement of the skin status quo by understanding how the intrinsic factors contribute to skin health and appearance. In an ideal world, Skin status quo should be equal to the DNA test predictions for the same set of skin characteristics. However, external factors play a major role and monitoring the intrinsic factors enables recommendations that are likely to change the status quo. Recommending products then becomes a quantifiable science, where both factors can be analysed and addressed. A further example of how the scores are useful is in assisting a physician in prioritizing which interventional treatments should be performed and to monitor the subjects' improvements or where further treatments are required.

To illustrate further, for example, if an individual's intrinsic code indicates that wrinkling is a primary concern for the subject but the skin status quo indicates minimal wrinkling, it can be concluded that the current lifestyle choices being made by the subject do not need to be tailored for a better wrinkling outcome.

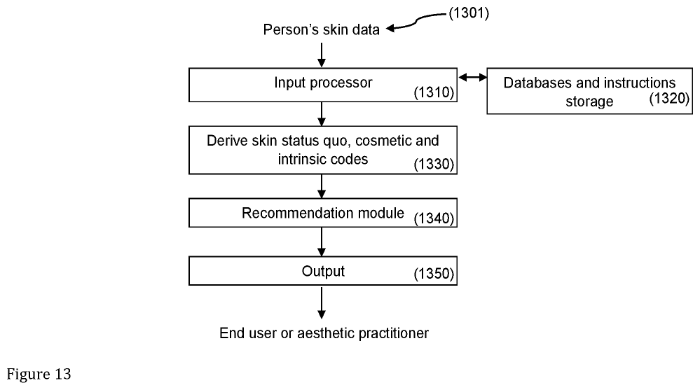

In a preferred embodiment of the disclosure includes a cosmetic system (1301) as exemplified in FIG. 13. The system may include at least a first processor (1310), a further processor may be included for integrating and analyzing the data received from the first processor to recommend a cosmetic recommendation as an output for a user (1340). The disclosure of this system has a computer readable storage medium (1320) for storing databases related to weighting tables, or cosmetic product databases and instructions to cause the processor to execute instructions to derive either a skin status quo code (1320), a cosmetic code or an intrinsic code (1330) and/or associated recommendations for a cosmetic product based on a cosmetic code are an output of the system (1350) to an aesthetic practitioner.

In a preferred example, a first processor may be a skin-scoring module (1410), a genetic scoring module (1519), and a cosmetic analyser (1420) and a further processor may be a recommendation module (1340) of FIG. 13 (and also denoted as (1450) in FIG.. 14 and 15).

In a preferred embodiment of a system is a skin-scoring module for deriving a skin status quo code (1410), a cosmetic analyser for deriving a cosmetic code for a cosmetic product and creating a weighted database of cosmetic products (1420), a processing and analyzing system for recommending a cosmetic code based on a skin status quo code (1450) and may include an output to an end user (1455) which may be a physician/aesthetic practitioner or an individual.

In the example of the skin-scoring module (1410), the system may include a data input medium that receives data related to a person's skin characteristics. The data may be a manual input from a practitioner after an evaluation of a person, (or may be a photographic image or video images), which may be, derived the method illustrated in FIG. 1 and specifically may be from any one or all steps (11) to (13). The data input may also be a visual image in the form of a photographic image or video images of a person or input data may be data derived from processing and related to specific predetermined skin characteristics. The skin-scoring module further has a means to process and assess the data input (1412) on the basis of a set of weight related tables (1415) to apply scores for each of the predetermines skin characteristics (1413) and to derive a skin status quo code (1414).

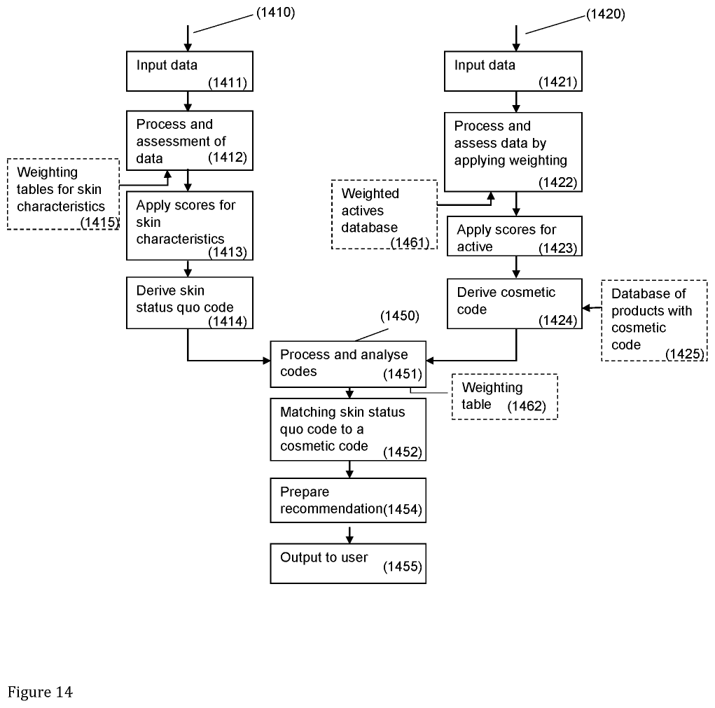

In the illustration of FIG. 14 is an example of a cosmetic analyser (1420), which is a processor for deriving a cosmetic code. Data input (1421) is a range of cosmetic products whose actives are known. Which is followed by the step of processing and assessing the data by applying weighting on the basis of the ability of an active to effect a skin characteristic (1422) based on the weighted actives database (1461), the results of the step (1422) are utilised to apply scores based on the weighting table to the actives (1423) and a further step of deriving a cosmetic code (1424). The step of deriving a cosmetic code, may include creating a sum of the scores as code for each of the skin characteristics and then aligning the skin characteristics on the basis of its descending order of impact of actives on a skin characteristics and a cosmetic code similar to code derived from the method of deriving cosmetic code (95). A further embodiment of the system is a database of cosmetic code derived cosmetic products (1425) and may be weighted (1462).

In a further aspect of the embodiment of FIG. 14, is a recommendation module (1450). In this example, the recommendation module processor is capable of receiving, processing and analyzing the skin status quo code and cosmetic code based cosmetic products (1414) to match the skin status quo code to at least a suitable cosmetic code (1452), which is chosen from the weighted table (1462) of cosmetic code based cosmetic products database. The recommendation module is further capable of executing instructions to prepare recommendations of specific cosmetic code based products and preparing and processing an output to an end user which may be the individual subject or an aesthetic practitioner.

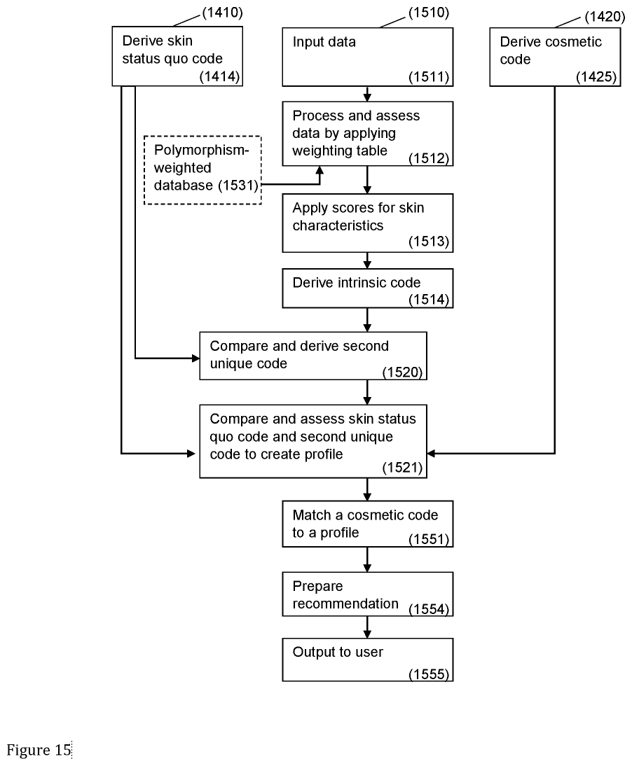

In another embodiment (FIG. 15), the system further comprises a processor, which is a genetic profiling module, wherein data is input into the system (1511), the input data is processed and assessed on the basis of a polymorphism weighted database or table (1531), wherein the risk of genotype variants for a particular skin category and therefore its effect on a skin characteristic are utilised for the weighting, scores are assigned for skin characteristics (1513) based on the weighting and an intrinsic code is derived as a cumulative weighted score (1514).

A recommendation module has a means to compare the skin status quo code and the intrinsic score for the individual (1520), such comparison to derive a second unique code. In a further step of the system, the second unique code and skin status quo code are compared and analysed to create a profile (1521), this profile may be utilised in matching a cosmetic code for the individual (1522) and the system is capable of making a recommendation based on the database of cosmetic products that is weighted (1554) and the recommendation may be the form of an output to a user.

It is a desirable object of this disclosure that the system is operable remote from an aesthetic practitioner, is capable of storing data related to individuals including profiles, which may include the skin status quo code based analysis of the second unique code. The system may be capable of storing and analyzing the data via a network server. It is possible that an embodiment of the system may not utilise weighted tables, but that the method of weighting and scoring is an artificial intelligence based system.

The Inventors believe that the invention as exemplified is advantageous in that the described method and system derived from the skin analysis can include both recommendations for cosmetic interventional treatments as well as skin care to use daily to improve the long-term outcome. Furthermore, the status quo and intrinsic codes will help individuals understand the cosmetic intervention recommendations as well as expected improvements to areas of concern. This systematic, defined, reproducible cosmetic intervention tool aims to bridge the gap between various treatment modalities and sustained skin health. Cosmetic interventions can include products that can quickly be developed to use in conjunction with emerging aesthetic treatment modalities. Currently as there exists no coherent offering of cosmetic interventions that extend from before an aesthetic treatment to beyond the post-treatment phase, a distinct advantage is that aesthetic cosmetic intervention can complement the results of invasive and non-invasive aesthetic treatments and can work alongside medical skin professionals to enable skin rejuvenation and work towards actively addressing them. This invention aims to profile skin systematically and comprehensively treat skin concerns and maintain the results of professional invasive and non-invasive interventions.

1. A cosmetic method, the method comprising the steps of:
dividing, at least one area of a person's skin into a plurality of zones;
evaluating, at least one of the plurality of zones for, at least one predetermined skin characteristic in each zone;
scoring, the zones on a predetermined scale of scores for, each predetermined skin characteristic based on the evaluation;
determining a collective score, wherein the scores for each zone for the predetermined skin characteristic is combined; and
assigning a skin status quo code, by utilizing the collective score for each predetermined skin characteristic.

2. The cosmetic method according to claim 1 wherein the step of scoring the zones on a predetermined scale of scores for each of the predetermined skin characteristics, is used to determine a zone score, which is a collective code of each of the scores of each of the predetermined areas within a zone; and the skin status quo code, is assigned by cumulating all zone scores as an overall severity ranking for the predetermined skin characteristic.

3. The cosmetic method according to claim 1 wherein the area of the person's skin is selected from the person's face, neck and/or a chest, which is divided into a minimum of 2 zones, a maximum of 15 zones, and may be 5 zones.

4. (canceled)

5. The cosmetic method according to claim 1 wherein at least one or more of the predetermined skin characteristics is selected from, the group consisting essentially of a characteristic of wrinkles, a characteristic of dryness, a characteristic of sensitivity and a characteristic of pigmentation.

6. The cosmetic method according to claim 1 wherein the step of evaluating the predetermined skin characteristics is based on a plurality of skin analysis techniques.

7. A cosmetic method, the method comprising the steps of:
choosing, a plurality of actives that has at least one remedial mode of action for at least one predetermined skin characteristic;
determining a score, from a range of predetermined scale of scores, for at least one of the plurality of actives based on the at least one remedial mode of action of the active;
combining, the scores of each of the actives based on the remedial mode of action, for the predetermined skin characteristic to assign a combined score for the predetermined skin characteristic; and
assigning a cosmetic code, to a cosmetic that contains the plurality of actives, based on the combined scores and directed to effect the at least one predetermined skin characteristic.

8. The cosmetic method according to claim 7 wherein the plurality of predetermined actives having remedial modes of action for the characteristics of wrinkles, dryness, sensitivity and pigmentation is selected from the group essentially consisting of anti-acne agents, antimicrobial agents, anti-inflammatory agents, analgesics, antietythemal agents, antipruritic agents, antiedemal agents, antipsoriatic agents, antifungal agents, skin protectants, sunscreen agents, vitamins, antioxidants, scavengers, anti-irritants, antibacterial agents, antiviral agents, anti-aging agents, photoprotection agents, hair growth enhancers, hair growth inhibitors, hair removal agents, antidandruff agents, anti-seborrheic agents, exfoliating agents, wound healing agents, anti-ectoparacitic agents, anti-pigmentation agents, whiteners, moisturizing agents, sebum modulators, immunomodulators, hormones, botanicals, moisturizers, astringents, cleansers, sensates, antibiotics, anesthetics, steroids, tissue healing substances, tissue regenerating substances, amino acids, peptides, minerals, ceramides and biohyaluronic acids.

9. The cosmetic method according to claim 1 wherein the method includes the steps of:
determining the at least one predetermined skin characteristic that may benefit from a cosmetic intervention, utilizing the status quo code; and
recommending at least one cosmetic intervention to effect the predetermined skin characteristic.

10. The cosmetic method according to claim 9 wherein the cosmetic intervention is a cosmetic code based product.

11. The cosmetic method according to claim 1, wherein the method includes the steps of:
determining an intrinsic code of the person's skin, for the predetermined skin characteristics wherein the intrinsic code of the person's skin, for the predetermined skin characteristics wherein the intrinsic code is derived by assigning scores for polymorphisms based on a priority code assigned to a presence or absence of a plurality of predetermined polymorphisms, which are known to affect the predetermined skin characteristics, selected from the group essentially consisting of MMP1, COL1A1, MC1R, ASIP, XRCC1, hOGG1, TERT, SOD2, GPX1, NQO1, GSTP1, EPHX1, IL6, IL6-R, CRP and TNFA genes;
comparing the intrinsic code, which is a measure of the effect of a person's genetics on the person's skin for predetermined skin characteristics to the skin status quo code;
deriving a second unique code based on the comparison, which represents an effect on predetermined skin characteristics that are not affected by the genetics of the person; and
recommending a cosmetic intervention directed to the second unique code, to effect the overall skin status quo code to effect at least one of the predetermined skin characteristics.

12. The cosmetic method according to claim 11 wherein the cosmetic intervention is a cosmetic code based product, wherein the cosmetic code for a cosmetic is derived by the steps of:
choosing, a plurality of actives that has at least one remedial mode of action for at least one predetermined skin characteristic;
determining a score, from a range of predetermined scale of scores, for at least one of the plurality of actives based on the at least one remedial mode of action of the active;
combining, the scores of each of the actives based on the remedial mode of action, for the predetermined skin characteristic to assign a combined score for the predetermined skin characteristic; and
assigning a cosmetic code, for a cosmetic that contains the plurality of actives, based on the combined scores and directed to effect the at least one predetermined skin characteristic.

13. A cosmetic system, the system comprising:
at least one processor to derive a cosmetic analysis data; and
a computer readable storage medium having one or more stored databases and instructions therein which, when executed by the processor, cause the processor to perform operations comprising the method of claim 1.

14. The cosmetic system according to claim 13 wherein the processor is a skin-scoring module and comprises a first input interface adapted to receive data of a plurality of predetermined skin characteristics; wherein the first input interface is adapted to receive a direct data input, data derived from analysis of a photographic image or video images of a zone or an area of a person's body in respect of at least one of a plurality of skin characteristics, and is adapted to process an image to derive data and receive that data as input.

15. (canceled)

16. (canceled)

17. The cosmetic system according to claim 14 wherein the skin-scoring module comprises:
means for, evaluating the data received by the first interface in relation to a weighting table for at least one of a plurality of predetermined skin characteristics;
means for scoring the zones on a predetermined scale of scores for each skin characteristic;
means for determining a collective score, wherein the scores for each zone or area of the body for the predetermined skin characteristic is combined; and
means for deriving the cosmetic analysis data, which is a status quo code by utilizing the collective score for each predetermined skin characteristic.

18. (canceled)

19. The cosmetic system according to claim 13 wherein the processor, is a cosmetic analyzer; comprising a second input interface adapted to receive data related to a plurality of actives in a cosmetic product.

20. The cosmetic system according to claim 19 wherein the cosmetic analyzer includes:
means for evaluating data received by the second input interface for the plurality of actives, for at least one remedial mode of action of the active, for at least a predetermined skin characteristic including;
means for determining a score, from a range of predetermined scale of scores utilizing a weighted actives database, for at least one of the plurality of actives based on the remedial modes of action of the active;
means for combining the scores of each of the actives for each predetermined skin characteristic to assign a combined score for the predetermined skin characteristic; and
means for deriving the cosmetic analysis data, which is a cosmetic code for a cosmetic product having the specific plurality of actives based on the combined score and directed to effect the at least one pre-determined skin characteristic.

21. The cosmetic system according to claim 20 wherein the means for evaluating the data received by the second input interface for the plurality of actives in a specific cosmetic product utilizes utilises data from a weighted actives database, which is stored in the computer readable storage medium.

22. The cosmetic system according to claim 13 wherein the processor includes a genetic scoring module, which includes a third input interface adapted to receive data related to a genetic polymorphism profiling, and
means for assigning specific scores for a plurality of predetermined skin characteristics from data obtained in the third input interface and utilizes a polymorphism weighting table to evaluate risks associated with a specific polymorphism;
means for deriving the cosmetic analysis data, which is an intrinsic code based on the assigned specific scores; and
means for comparing the intrinsic code to the skin status quo code, utilizing a predictive equation directed to deriving further cosmetic analysis data, a second unique code, to represent effects on predetermined skin characteristics which excludes those attributable to the genetics of a person.

23. (canceled)

24. The cosmetic system according to claim 13 wherein the processor includes, a recommendation module which is adapted to receive a plurality of data related to the cosmetic analysis data; and comprises a means for integrating cosmetic analysis data, wherein the recommendation module includes:
means for analyzing the skin status quo code derived from the system;
means for executing a predictive equation, for determining the at least one predetermined skin characteristic that can benefit from a cosmetic code based cosmetic product; and
means for integrating a recommendation of a cosmetic product with a specific cosmetic code to effect at least one predetermined skin characteristic.

25. (canceled)

26. The cosmetic system of claim 13 wherein the computer readable storage includes:
databases which include a plurality of weighting tables, wherein the weighting table database is selected from the group including a weighting of scores for the predetermined skin characteristics,
a weighted actives database,
a polymorphism weighting database,
a cosmetics products database of cosmetic products wherein a set of cosmetic coded products are weighed on the basis of the ability of its plurality of actives to effect a predetermined skin characteristic; and
a set of instructions directed to at least one of a plurality of predictive equations for deriving a skin status quo code, an intrinsic code, a cosmetic code, a second unique code and/or a set of instructions to utilize a selection of the codes to make a cosmetic recommendation.

27. (canceled)

28. (canceled)

29. (canceled)

30. (canceled)

31. (canceled)

32. (canceled)
